(19) United States
(12) Patent Application Publication
(10) Pub. No.: US 2022/0273671 A1
(43) Pub. Date: Sep. 1, 2022

(54) TOPICAL FORMULATIONS FOR TREATMENT OF PERIPHERAL NEUROPATHIES

(71) Applicant: WinSanTor, Inc., San Diego, CA (US)

(72) Inventors: Angela Hansen, San Diego, CA (US); Edward Kisak, San Diego, CA (US); John M. Newsam, San Diego, CA (US)

(21) Appl. No.: 17/593,792

(22) PCT Filed: Mar. 24, 2020
(86) PCT No.: PCT/US2020/024504

§ 371 Date: Sep. 24, 2021

Related U.S. Application Data
(60) Provisional application No. 62/824,060, filed on Mar. 26, 2019.

(51) Int. Cl. A61K 31/5513; A61K 45/06; A61K 31/4415; A61K 31/51; A61K 31/525; A61K 31/714; A61K 33/06; A61K 47/02; A61K 47/10; A61K 47/12; A61K 47/14; A61K 47/20; A61K 47/22; A61K 9/00; A61K 9/10; A61P 25/02

(52) U.S. Cl. CPC A61K 31/5513; A61K 45/06; A61K 31/4415; A61K 31/51; A61K 31/525; A61K 31/714; A61K 33/06; A61K 47/02; A61K 47/10; A61K 47/12; A61K 47/14; A61K 47/20; A61K 47/22; A61K 9/0014; A61K 9/10; A61P 25/02

(57) ABSTRACT

Aspects relate to topical formulations of a muscarinic acetylcholine receptor antagonist or a salt or derivative in combination with DMSO and a polyalkylene glycol alkyl ether. The topical formulations may include pirenzepine as the muscarinic acetylcholine receptor antagonist. In addition, the polyalkylene glycol alkyl ether may be a polyethylene glycol alkyl ether.

### PRIORITY AND CROSS-REFERENCE TO RELATED APPLICATIONS

This application claims the benefit of U.S. Provisional Application 62/824,060, filed on Mar. 26, 2019, which is hereby incorporated by reference in its entirety.

### FIELD OF THE INVENTION

This invention relates to therapies for treatment of peripheral neuropathies in a patient. More particularly, this invention relates to therapeutic compositions for peripheral neuropathy, wherein the compositions comprise an effective amount of a topical muscarinic acetylcholine receptor antagonist.

### BACKGROUND

Peripheral neuropathy is a condition involving functional and/or structural damage to the peripheral nervous system. Peripheral neuropathy generally refers to a disorder that affects one or a combination of the motor, sensory or autonomic nerves. Numbness, tingling, pain, weakness, muscle cramping, twitching, and erectile dysfunction are common complaints. The wide variety of disorders exhibited by peripheral neuropathies can each be uniquely attributed to an equally wide variety of causes. For instance, peripheral neuropathies can be genetically acquired, can result from a systemic disease, an infectious disease (e.g., viral or bacterial), can manifest as a post-surgical complication, or can be induced by a toxic agent. Some toxic agents that cause neurotoxicities are therapeutic drugs, antineoplastic agents, contaminants in foods or medicines and environmental and industrial pollutants. Three percent or more of the human population may be affected by peripheral neuropathy.

Peripheral neuropathies include the following: neuropathy associated with diabetes mellitus (diabetic neuropathy), HIV-associated neuropathy; nutritional deficiency-associated neuropathy; cranial nerve palsies; drug-induced neuropathy; industrial neuropathy; lymphomatous neuropathy; myelomatous neuropathy; multi-focal motor neuropathy; immune-mediated disorders, chronic idiopathic sensory neuropathy; carcinomatous neuropathy; acute pain autonomic neuropathy; alcoholic neuropathy; compressive neuropathy; vasculitic/ischaemic neuropathy; mono- and polyneuropathies. Other peripheral neuropathies may arise from Raynaud's Phenomenon (including CREST syndrome), leprosy and autoimmune diseases such as erythromatosis and rheumatoid diseases.

Some neuropathies are caused by toxic agents. These toxic agents may include therapeutic agents, particularly those used to treat neoplastic diseases. In certain cases, peripheral neuropathy is a major complication of cancer treatment and a main factor limiting the dosage of chemotherapeutic agents that can be administered to a patient. Treatment options for patients having peripheral neuropathies are currently limited, with most methods generally consisting of analgesics for managing pain, for example to decrease the perception of pain, decrease reactions to pain, or to increase pain tolerance.

### SUMMARY OF THE INVENTION

Accordingly, embodiments relate to treatment of peripheral neuropathies with topical formulations of muscarinic M1 receptor antagonists, such as pirenzepine. In some embodiments, the formulations comprise a muscarinic receptor subtype 1 antagonist, and a topical vehicle. The topical vehicle may include a low volatility solvent and a polyether surfactant.

Some embodiments relate to a topical formulation. In some embodiments, the topical formulation includes (i) a muscarinic acetylcholine receptor antagonist or a salt or derivative, (ii) DMSO, and/or (iii) a polyalkylene glycol alkyl ether. In some embodiments, the muscarinic acetylcholine receptor antagonist is pirenzepine, pirenzepine free base, or a pirenzepine salt. In some embodiments, the polyalkylene glycol alkyl ether is a polyethylene glycol alkyl ether. Some embodiments include a fatty acid ester. In some embodiments, the fatty acid ester is lauryl lactate. Some embodiments include benzyl alcohol. Some embodiments include dimethyl isosorbide. In some embodiments, the muscarinic acetylcholine receptor antagonist is a pirenzepine salt or pirenzepine free base, and the composition comprises less than 10% pirenzepine salt or pirenzepine free base, respectively. Some embodiments include about 1% to 5% pirenzepine salt or pirenzepine free base. In some embodiments, the topical formulation is a topical gel formulation.

Some embodiments relate to a method of treating peripheral neuropathy in a subject. Some embodiments of the method include topically administering a topical formulation as described herein to the subject. In some embodiments, the topical formulation is administered once daily. In some embodiments, the topical formulation is administered in a first daily dosage for the first seven days and a second daily dosage for at least the next seven days, wherein the second daily dosage comprises twice as much formulation as the first daily dosage. In some embodiments, the first dosage is 2.5 mL of the topical formulation and the second dosage is 5.0 mL of the topical formulation.

### BRIEF DESCRIPTION OF THE DRAWINGS

The embodiments of the application will now be described in greater detail with reference to the attached drawings in which:

FIG. 1 is a bar chart showing percent delivery of pirenzepine in varying solvents in a water-based vehicle. The flux study was done using porcine skin as the substrate. Percent delivery indicates the percent of the applied dose of pirenzepine dihydrochloride that crossed the skin over 24 hours.

FIG. 2 is a bar chart showing the percent delivery of pirenzepine in varying surfactants in a water with DMSO-based vehicle. The study was done using porcine skin as the substrate. Percent delivery indicates the percent of the applied dose of pirenzepine dihydrochloride that crossed the skin over 24 hours.

FIG. 3 is a bar chart showing the percent delivery of pirenzepine with various penetration enhancers in a water/DMSO/dimethyl isosorbide/Brij 78 base. The study was done using porcine skin as the substrate. Percent delivery indicates the percent of the applied dose of pirenzepine dihydrochloride that crossed the skin over 24 hours.

FIG. 4 is a bar chart showing the percent delivery of pirenzepine in varying excipients which were added to a water based vehicle. As shown, there was a stepwise increase the flux rate of pirenzepine dihydrochloride across skin. The flux study was done using porcine skin as the substrate. Percent delivery indicates the percent of the applied dose of pirenzepine dihydrochloride that crossed the skin over 24 hours.

FIG. 5 is a bar chart showing the delivered dose of varying formulations as compared to a control gel to determine the effectiveness of different penetration enhancers in a gel format. The flux study was done using cadaver skin as the substrate. Results are shown as total delivered dose in μg/cm2. Epidermal and dermal concentrations were taken at the end of the experiment, after approximately 20 hours.

FIG. 6 is a bar chart showing the delivered dose of varying formulations as compared to a control gel to determine the effectiveness of different penetration enhancers in a gel format across different skin layers. WinF54 was not gelled and left as a liquid. The flux study was done using cadaver skin as the substrate. Results are shown as total delivered dose in μg/cm2. Epidermal and dermal concentrations were taken at the end of the experiment, after approximately 20 hours.

FIG. 7 is a bar chart showing varying pirenzepine free base formulations that were compared against a pirenzepine dihydrochloride gel. Results are shown as total delivered dose in μg/cm2. Epidermal and dermal concentrations were taken at the end of the experiment, after approximately 40 hours.

FIG. 8 is a line graph showing the amount of pirenzepine in plasma over time. The entire (Mean) Pirenzepine Plasma Profile by Cohort where: “1” is formulation WinFB34; “2” is formulation WinF90, and “3” is formulation WinFB100.

FIG. 9 is a bar chart showing the concentration of pirenzepine in participants in Cohort 2, using formulation WinF90. The biopsies were taken from the left calfskin, at an upper or lower location.

FIG. 10 is a line graph showing the concentration of pirenzepine in plasma over time after receiving a dose of formulation WinF90. Data in this figure are taken from members of Cohort 2 after being treated with formulation WinF90 for 14 days.

FIG. 11 is a bar chart showing the concentration of pirenzepine in participants in Cohort 3, using formulation WinFB100. The biopsies were taken from the left calfskin, at an upper or lower location.

FIG. 12 is a line graph showing the concentration of pirenzepine in plasma over time after receiving a dose of formulation WinFB100. Data in this figure are taken from members of Cohort 3 after being treated with formulation WinFB100 for 14 days.

FIG. 13 is a bar chart showing skin biopsy results for untreated skin in Cohort 2 (the cohort that received WinF90).

FIG. 14 is a bar chart showing skin biopsy results for untreated skin in Cohort 3 (the cohort that received WinFB100).

FIG. 15 shows a bar graph of the total delivered dose (in μg/cm²) in skin biopsies at 22 hours using embodiments of formulations disclosed herein.

FIG. 16 shows a graph of the total delivered dose (in μg/cm²) in skin biopsies at 4 hours and 20 hours using embodiments of formulations disclosed herein, and a comparison of retention in the epidermis and dermis layers of the skin.

FIG. 17 shows a graph of the total delivered dose (in μg/cm²) of pirenzepine via the transdermal route in skin biopsies at 3 hours, 6 hours, and 24 hours using embodiments of formulations disclosed herein, and a comparison of retention in the epidermis and dermis layers of the skin.

FIG. 18 shows a graph of the total delivered dose (in μg/cm²) of pirenzepine via the transdermal route in skin biopsies at 3 hours, 6 hours, and 24 hours using embodiments of formulations disclosed herein, and a comparison of retention in the epidermis and dermis layers of the skin.

### DETAILED DESCRIPTION OF THE INVENTION

Embodiments relate to topical pharmaceutical compositions for treating peripheral neuropathy and comprising a muscarinic receptor subtype 1 antagonist, and a topical vehicle. In some embodiments, the topical vehicle includes a low volatility solvent and a polyether surfactant.

The composition may be used for the manufacture of a medicament for the treatment or prevention of peripheral neuropathy.

A further embodiment of the invention relates to a method for treating a subject afflicted with peripheral neuropathy which comprises applying to the affected area of skin of said subject an effective amount of a composition as defined above.

In one embodiment, the method of using the topical pharmaceutical composition of the invention is by applying it to completely cover the affected area. The frequency of the application can be periodic or recurring, for example, daily, although adequate maintenance therapy for some patients may be achieved with less frequent application.

Some embodiments relate to a topical formulation or pharmaceutical composition that does not penetrate through the skin in high amounts into the blood and/or systemically. For example, topical administration of the topical pharmaceutical composition of some embodiments results in little, minimal or no exposure of a medicament (such as, but not limited to pirenzepine) of the topical pharmaceutical composition into the blood of a subject to which the topical pharmaceutical composition is topically applied. As used herein, minimal or little exposure refers to less than 30 nanograms per ml concentration of the medicament, or less than 20 nanograms per ml concentrations of the active ingredient as measured in the blood or systemically. In one embodiment, minimal or little exposure refers to 15, 14, 13, 12, 11, 10, 9, 8, 7, 6, 5, 4, 3, 2, or 1 nanogram per ml concentrations of the active ingredient as measured in the blood or systemically. Some such embodiments relate to a method of topically applying the topical pharmaceutical composition without resultant systemic administration of a medicament of the topical pharmaceutical composition. In some other embodiments, topical administration of a topical pharmaceutical composition results in systemic administration of a medicament of the topical pharmaceutical composition.

In some embodiments, topical administration of the topical pharmaceutical composition results in a 10-20%, 20-30%, 30-40%, 40-50%, 50-60%, 60-70%, 70-80%, 80-90%, 90-95%, or 95-100% of a medicament of the topical pharmaceutical composition being retained in or captured by the skin (without entering the bloodstream, for example) of the subject to which the topical pharmaceutical composition is topically applied. in some embodiments, topical administration of the topical pharmaceutical composition to an area other than the skin results in a most or all of the medicament of the topical formulation being retained or captured by the area where the topical formulation is applied. Thus, some embodiments further relate to a topical pharmaceutical composition that is safely administered to a patient or subject, and is retained at or near the site where the topical pharmaceutical composition is topically applied without traveling through the bloodstream of the patient or subject. In some embodiments, the medicament of the topical pharmaceutical composition travels through the interstitium of the subject upon topical administration of the topical pharmaceutical composition to the subject. In other embodiments, the medicament of the topical pharmaceutical composition does not travel through the interstitium of the subject upon topical administration of the topical pharmaceutical composition to the subject.

It was previously discovered that culturing excised adult and juvenile neurons from normal and diabetic rats, in culture media containing M1 antagonists stimulate neurite outgrowth from the excised neurons. Additionally, topical applications of pirenzepine to and into various skin targets in diabetic mice: (i) prevented deficits in motor nerve conduction velocity, (ii) prevented and reversed loss of intraepidermal nerve fibers, (iii) prevented loss of sub-epidermal nerve plexus, (iv) prevented tactile allodynia, and (v) prevented and reversed the development of thermal hypoalgesia. See for example, PCT Application Nos. WO/2012/055018A1, Therapeutic Compositions For Diabetic Symmetrical Polyneuropathy; and WO/2015/089664A1, Methods And Compositions For Treatment Of Peripheral Neuropathies.

A number of selective antagonists for the type 1 muscarinic receptor (M1R) that discriminate one subtype of muscarinic receptor subtypes (e.g., M1R) versus other subtypes were shown to have similar affects. More generally, muscarinic acetylcholine receptor antagonists appear to reverse loss of intraepidermal nerve fibers and thermal hypoalgesia.

As used herein, muscarinic acetylcholine receptor antagonists are agents that reduce the activities and/or function of muscarinic acetylcholine receptors that are found in the plasma membranes of neurons and other cells. Muscarinic acetylcholine receptors are stimulated by acetylcholine released from several cell types including sensory neurons, postganglionic fibers in the parasympathetic nervous system and keratinocytes that function as signaling molecules to initiate signal cascades within cells in their immediate regions. Well-known muscarinic acetylcholine receptor antagonists useful for treatment of maladies such as central nervous system malfunctioning, pulmonary diseases, and gastric ailments are exemplified by atropine, scopolamine, telenzepine, hyoscine, hyoscyamine, ipratropium, tropicamide, cyclopentolate, glycopyrrolate, 4-diphenylacetoxy-1,1-dimethylpiperidinium, o-methoxy-sila-hexocyclium, quinidine, Oxyphenonium, orphenadrine, oxybutynin, oxyphenonium, emepronium, metixene, procyclidine, propantheline, 4-fluorhexahydrosiladifenidol, octylonium, quinuclidinyl benzilate, buclizine, clidinium, Cyproheptadine, imidafenacin, chlorpromazine, cycrimine, doxepin, thorazine, doxylamine, escitalopram, flupentixol, methantheline, mepenzolate, haloperidol, tolterodine, benactyzine, benzatropine, fesoterodine (fumarate), ethopropazine, trospium, solifenacin, gallamine, biperiden, dicyclomine, dexetimide, hexahydrosiladifenidol, VU 0255035, among others.

In one embodiment, the present invention relates to tricyclic muscarinic subtype 1 (M1) antagonists, particular a heterocyclic compound containing an azepine or diazepine ring fused to a benzene ring and a 5 or 6 membered aromatic ring such as second benzene ring, thiophene ring, pyrrole ring, imidazole ring, pyrazole ring, pyridine ring, pyrazine ring, pyrimidine ring, or a pyridazine ring. Such compounds include dibenzazepines (iminostilbene) or dibenzadiazepines, thienobenzodiazepines such as telenzepine, or a pyridine benzodiazepines such as pirenzepine, and other M1 antagonists. In one embodiment, the present invention relates to M1 selective antagonists that have greater selectivity for the muscarinic subtype 1 receptor.

Several selective M1 antagonists were found to exhibit beneficial activities similar to those demonstrated by pirenzepine or telenzepine on neuronal neurite outgrowth. Some of these compounds have greater M1R selectivity. For example, telenzepine, is an analog of pirenzepine with an altered tricyclic structure but an unmodified piperazine side chain. VU0255035, a thiadiazole derivative and is 75 times more selective to M1R relative to M2, M3, M4 and M5 receptors. Among the new generation of M1R antagonists, there are several promising centrally active M1R antagonists, including PD150714 and spirotramine.

Typically, the M1 antagonist concentration is in the range of 0.01 wt. % to 20.0 wt. %, or for example, in the range of 0.5 wt. % to 15 wt. %, or in the range of 1.0 wt. % to 10 wt. %, based on the total weight of the composition.

According to one embodiment of the invention, the M1 antagonist is pirenzepine or a salt thereof.

According to one embodiment of the invention, the pirenzepine or a salt thereof is in the form of pirenzepine free base.

The pirenzepine free base concentration may be in the range of 0.01 wt. % to 20.0 wt. %, or for example, in the range of 0.5 wt. % to 15 wt. %, or in the range of 1.0 wt. % to 10 wt. %, based on the total weight of the composition. The pirenzepine free base concentration may also be in the range of 0.1 wt. % to 5.0 wt. %, or for example, in the range of 0.25 wt. % to 3 wt. %, or 1 wt. % to 2 wt. %, based on the total weight of the composition. In some embodiments, the pirenzepine free base concentration is approximately 1, 2, 3, 4, 5, 6, 7, 8, 9, 10, 11, 12, 13, 14, 15, 16, 17, 18, 19, or 20 percent of the final concentration by weight of the formulation. In other embodiments, the pirenzepine free base concentration may be less than or equal to 1, 2, 3, 4, 5, 6, 7, 8, 9, 10, 11, 12, 13, 14, 15, 16, 17, 18, 19, or 20 percent of the final concentration by weight of the formulation.

According to one embodiment of the invention, the pirenzepine or a salt thereof is in the form of pirenzepine dihydrochloride salt.

In some embodiments, the pirenzepine dihydrochloride salt concentration is in the range of 0.01 wt. % to 20.0 wt. %, or for example, in the range of 0.5 wt. % to 15 wt. %, or in the range of 1.0 wt. % to 10 wt. %, based on the total weight of the composition. In other embodiments, the pirenzepine dihydrochloride salt concentration is in the range of 0.1 wt. % to 5.0 wt. %, or for example, in the range of 0.25 wt. % to 3 wt. %, or 1 wt. % to 2 wt. %, based on the total weight of the composition. In some embodiments, the pirenzepine dihydrochloride salt concentration is approximately 1, 2, 3, 4, 5, 6, 7, 8, 9, 10, 11, 12, 13, 14, 15, 16, 17, 18, 19, or 20 percent of the final concentration by weight of the formulation. In other embodiments, the pirenzepine dihydrochloride salt concentration may be less than or equal to 1, 2, 3, 4, 5, 6, 7, 8, 9, 10, 11, 12, 13, 14, 15, 16, 17, 18, 19, or 20 percent of the final concentration by weight of the formulation.

As used herein, the term “neuropathy” includes any pathology or abnormality of neural tissue causing nerve dysfunction. The function of the nerve or nerves that is disrupted may involve the rate of flow of the electrical current through the nerve or may involve ectopic firing (firing in the absence of stimulus) of the nerve, or may involve inappropriate or inadequate firing of the nerve in response to a stimulus. Peripheral neuropathy as used herein is defined as a disorder resulting from damage to peripheral nerves. It may be acquired, caused by diseases of the nerves or as the result of systemic illness.

A number of factors can cause, induce or are associated with peripheral neuropathies, and included within the scope of the invention are peripheral neuropathies associated with, diabetes, infectious disease, toxic agents, chemotherapeutic agents, alcoholism, nutritional-deficiencies, systemic/metabolic disorders, palsies, auto-immune disorders, inherited or genetic disorders, cancers and tumors, compressive neuropathies; vasculitic/ischaemic neuropathy; mono- and polyneuropathies. Peripheral neuropathy has also been associated with erectile dysfunction. In further embodiments, the peripheral neuropathy manifests as a post-surgical complication.

Included within the scope of the term neuropathy/neuropathies are neuropathies associated with diseases such as: uremia; childhood cholestatic liver disease; chronic respiratory insufficiency; alcohol; multiple organ failure; sepsis; hypoalbuminemia; eosinophilia-myalgia syndrome; hypoglycemia; vitamin or nutritional deficiency (e.g., B-12 deficiency, vitamin A deficiency, vitamin E deficiency, vitamin B1 deficiency); primary biliary cirrhosis; hyperlipidemia; sensory perineuritis; allergic granulomatous angiitis; hypersensitivity angiitis; Bell's Palsy, Wegener's granulomatosis; rheumatoid arthritis; systemic lupus erythematosus; mixed connective tissue disease; scleroderma; systemic vasculitides; acute tunnel syndrome; pandysautonomia; hypothyroidism; chronic obstructive pulmonary disease; acromegaly; malabsorption (sprue, celiac disease); carcinomas (sensory, sensorimotor, late and demyelinating); lymphoma (including Hodgkin's), polycythemia vera; multiple myeloma (lytic type, osteosclerotic, or solitary plasmacytoma); tropical myeloneuropathies; pernicious anemia, Churg-Strauss syndrome; cranial nerve palsies; drug-induced neuropathy; industrial neuropathy; lymphomatous neuropathy; myelomatous neuropathy, chronic idiopathic sensory neuropathy; carcinomatous neuropathy; acute pain autonomic neuropathy; compressive neuropathy; mono- and polyneuropathies; or diabetes.

Diabetic neuropathies is one of the most common forms of diabetic peripheral neuropathy. Accordingly, one embodiment is diabetic peripheral neuropathy or diabetic neuropathy. It will be clearly understood that the diabetic neuropathy may be diabetic or pre-diabetic associated with Type 1 (insulin-dependent) diabetes, Type 2 (non-insulin-dependent) diabetes, or both.

In other embodiments the peripheral neuropathy is induced by, or a secondary affect due to, a toxic agent such as a drug, industrial chemical or environmental toxin. For example, the peripheral neuropathy can be caused by a chemotherapeutic agent such as paclitaxel (or other taxane derivatives), alkaloids such as vincristine or vinblastin, platinum compounds such as cis, carbo- and oxali-platin, topoisomerase inhibitors, intercalators such as bleomycin, or drugs such as chloramphenicol, colchicine, dapsone, disulfiram, amiodarone, gold, isoniazid, misonidazole, nitrofurantoin, perhexiline, propafenone, pyridoxine, phenytoin, simvastatin, tacrolimus, thalidomide, cyclophosphamide or zalcitabine, an agent used for the treatment of infectious diseases such as streptomycin, didanosine or zalcitabine, or any other chemically toxic agent such as acrylamide, arsenic, carbon disulfide, hexacarbons, lead, mercury, platinum, an organophosphate, thallium, or alcohol. Some other drugs that may cause peripheral neuropathy include:
Anti-alcohol drugs (Disulfiram)
AnticonvulsantsPhenytoin (Dilantin®)
Cancer medications (Cisplatin)
Vincristine
Heart or blood pressure medications (Amiodarone)
Hydralazine
Perhexiline
Infection fighting drugs (Metronidazole, Flagyl®, Fluoroquinolones: ciprofloxacin (Cipro®), gemifloxacin (Factive®), levofloxacin (Levaquin®), moxifloxacin (Avelox®), norfloxacin (Noroxin®), and ofloxacin (Floxin®).)
Nitrofurantoin
Thalidomide
TNF blockers (Humira®)
INH (Isoniazid)
Skin condition treatment drugs (Dapsone)

In another embodiment, the peripheral neuropathy caused by a systemic or metabolic disease is selected from the group consisting of diabetic or pre-diabetic neuropathy, acquired primary demyelinating neuropathy, distal symmetric sensory polyneuropathy, distal symmetric sensorimotor polyneuropathy, vasculitic neuropathy, infectious neuropathy, idiopathic neuropathy; immune-mediated neuropathy; nutrition-related neuropathy, kidney or liver failure, and paraneoplastic neuropathy.

In other embodiments, the peripheral neuropathy is induced by an infection or infectious disease, such as leprosy, Lyme disease, HIV or acquired immunodeficiency syndrome (AIDS)-associated neuropathy, post-polio syndrome, herpes simplex and herpes zoster (aka shingles); hepatitis B, hepatitis C, HIV, cytomegalovirus, or diphtheria.

In one embodiment, immune-mediated such as acquired primary demyelinating neuropathy includes chronic inflammatory demyelinating polyradiculoneuropathy (CIDP), Guillain-Barre syndrome/acute inflammatory demyelinating polyneuropathy (AIDP), sarcoidosis; vasculitic/ischaemic neuropathy (such as polyarteritis nodosa, rheumatoid arthritis, systemic lupus erythematosus (Lupus) and Sjogren's syndrome), celiac disease (sprue), multi-focal motor neuropathy (MNN), or peripheral neuropathy associated with protein abnormalities (such as monoclonal gammopathy, amyloidosis, cryoglobulinemia, macroglobulinemia, POEMS).

In one embodiment, the compression that causes peripheral neuropathy is selected from the group consisting of carpal tunnel syndrome, ulnar neuropathy at the elbow or wrist, common peroneal nerve at the knee, tibial nerve at the knee, amyloidosis, and sciatic nerve.

Also included within the scope of the term neuropathy/neuropathies are hereditary or genetically acquired neuropathies, including peroneal muscular atrophy (Charcot-Marie-Tooth Disease) hereditary amyloid neuropathies, hereditary sensory neuropathy (type I and type II), porphylias or porphyric neuropathy, hereditary (neuropathy) liability to pressure palsy (HNPP), Fabry's Disease, adrenomyeloneuropathy, Riley-Day Syndrome, Dejerine-Sottas neuropathy (hereditary motor-sensory neuropathy-III), Refsum's disease, Raynaud's disease including CREST syndrome. Krabbe's disease, ataxia-telangiectasia, hereditary tyrosinemia, anaphalipoproteinemia, abetalipoproteinemia, giant axonal neuropathy, metachromatic leukodystrophy, globoid cell leukodystrophy, or Friedrich's ataxia.

The compositions and methods of the invention can be also be used to treat or prevent neuropathy related to or induced by the following diseases, trauma, or conditions: general neuropathic conditions, such as peripheral neuropathy, phantom limb pain, reflex-sympathetic dystrophy, causalgia, syringomyelia, erectile dysfunction, and painful scar; specific neuralgias at any location of the body; back pain; diabetic neuropathy; alcoholic neuropathy; metabolic neuropathy; inflammatory neuropathy; chemotherapy-induced neuropathy, herpetic neuralgias; traumatic odontalgia; endodontic odontalgia; thoracic-outlet syndrome; cervical, thoracic, or lumbar radiculopathies with nerve compression; cancer with nerve invasion; traumatic-avulsion injuries; mastectomy, surgically-induced, thoracotomy pain; spinal-cord-injury; stroke; abdominal-cutaneous nerve entrapments; tumors of neural tissues; arachnoiditis; stump pain; fibromyalgia; regional sprains or strains; myofascial pain; psoriatic arthropathy; polyarteritis nodosa; osteomyelitis; burns involving nerve damage; frost-bite or other environmentally induced neuropathies, AIDS-related pain syndromes; connective tissue disorders, such as systemic lupus erythematosis, systemic sclerosis, polymyositis, and dermatomyositis; and inflammatory conditions, such as acute inflammation (e.g. trauma, surgery and infectious disease) or chronic inflammation (e.g., arthritis and gout).

As used herein, the term “carrier” or “vehicle” refers to carrier material suitable for topical drug administration and includes any such material known in the art, e.g. any liquid, gel, solvent, liquid diluent, solubilizer or the like, which does not interact with other components of the composition in a deleterious manner. Some carriers or vehicles may be selected from water, petroleum hydrocarbons, fatty acids, fatty acid esters, fatty alcohols, fatty alcohol ethers, fatty alcohol esters, C2-C8 linear or branched, saturated or unsaturated alcohols, polyols, aromatic alcohols, alkyleneglycol ethers, alkyleneglycol esters, natural waxes and silicones or mixtures thereof. Examples of suitable carriers can be found at Martindale—The complete drug reference, 38th edition, Pharmaceutical Press 2014.

In one embodiment, the composition includes a vehicle or carrier comprising a low volatility solvent and a polyether surfactant. In one embodiment, the low volatility solvent is an organic solvent, such as dimethyl sulfoxide (DMSO). In one embodiment, alternative or in addition to the previous embodiment, the polyether surfactant is a polyethylene glycol alkyl ether.

In one embodiment, the carrier or vehicle is selected from fatty acids, fatty acid esters, fatty alcohols, fatty alcohol ethers, fatty alcohol esters, C2-C8 linear or branched, saturated or unsaturated alcohols, polyols, aromatic alcohols, alkyleneglycol ethers, alkyleneglycol esters and natural or mixtures thereof.

The topical pharmaceutical compositions according to the invention may optionally further comprise other well-known pharmaceutically and/or cosmetically acceptable additives, such as, e.g. anti-irritants, antioxidants, buffering agents (pH adjusting agents), chelating agents, emollients, preservative agents, solubilizing agents, thickening agents, wetting agents, and the like, or mixtures thereof.

In some embodiments, low volatility components can be used in order to increase the mass median aerodynamic diameter (MMAD) to improve the solubility of the active ingredient in the system. In some embodiments, the low volatility component has typically a high boiling point (e.g., in the range of 150° C.-250° C.), and/or low vapor pressure (e.g., vapor pressure at 25° C. lower than 0.1 kPa, or lower than 0.05 kPa). Examples of low-volatility components include, but are not limited to: esters such as isopropyl myristate, ascorbyl myristate, tocopherol esters; glycols such as propylene glycol, polyethylene glycol, glycerol; and surface active agents such as saturated organic carboxylic acids (e.g., lauric, myristic, stearic acid) and unsaturated carboxylic acids (e.g., oleic or ascorbic acid). Examples include N-methylpyrrolidone (NMP), dimethylsulfoxide (DMS 0) or dimethylacetamide (DMAc), or 1,3-dimethyl-2-imidazolidinone (DMI), dimethylacetamide, dimethylformamide (DMF), hexamethyl phosphorotriamide (HMPT), ethylene glycol, polyethylene glycol, xylene, propylene glycol, pyrrolidone, glycerol, 1,4-dioxane, triethanolamine or mixtures thereof. isopropyl myristate, miglyol or glycerol.

The low volatility solvent can vary from 0.1 to 60% w/w, from 1 to 50% w/w, or from 10 to 40% w/w by weight.

Surfactants which can be used to form pharmaceutical compositions and dosage forms of the invention include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

One surfactant may be the sodium salt form of the compound, which may include the monosodium salt form. Suitable sodium salt surfactants may be selected based on desirable properties, including high speed of polymerization, small resultant particle sizes suitable for delivery, good polymerization yields, stability including freeze-thaw and shelf-life stability, improved surface tension properties, and lubrication properties.

The surfactant may be any suitable, non-toxic compound that is non-reactive with the medicament and that substantially reduces the surface tension between the medicament, the excipient and the site of administration. The surfactants include but are not limited to: oleic acid available under the tradenames Mednique 6322 and Emersol 6321 (from Cognis Corp., Cincinnati, Ohio); cetylpyridinium chloride (from Arrow Chemical, Inc. Westwood, N.J.); soya lecithin available under the tradename Epikuron 200 (from Lucas Meyer Decatur, Ill.); polyoxyethylene(20) sorbitan monolaurate available under the tradename Tween 20 (from ICI Specialty Chemicals, Wilmington, Del.); polyoxyethylene(20) sorbitan monostearate available under the tradename Tween 60 (from ICI); polyoxyethylene(20) sorbitan monooleate available under the tradename Tween 80 (from ICI); polyoxyethylene (10) stearyl ether available under the tradename Brij 76 (from ICI); polyoxyethylene (2) oleyl ether available under the tradename Brij 92 (from ICI); Polyoxyethylene-polyoxypropylene-ethylenediamine block copolymer available under the tradename Tetronic 150 R1 (from BASF); polyoxypropylene-polyoxyethylene block copolymers available under the tradenames Pluronic L-92, Pluronic L-121 end Pluronic F 68 (from BASF); castor oil ethoxylate available under the tradename Alkasurf CO-40 (from Rhone-Poulenc Mississauga Ontario, Canada); and mixtures thereof.

A suitable hydrophilic surfactant may generally have a hydrophilic-lipophilic balance (HLB) value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10oleate, Tween 40, Tween 60, sucrose monostearate, Kolliphor® HS 15, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

Surfactants may be used in any formulation of the invention where its use is not otherwise contradicted. In some embodiments of the invention, the use of no surfactants or limited classes of surfactants is desirable. The topical formulations according to the invention can contain no, or substantially no surfactant, i.e. contain less than approximately 0.0001% by weight of surface-active agents. This is particularly the case if one employs a cromone as described above. If desired, however, the formulations can contain surface-active agents conventionally employed in topical formulations, such as oleic acid, lecithin, sorbitan trioleate, cetylpyridinium chloride, benzalkonium chloride, polyoxyethylene (20) sorbitan monolaurate, polyoxyethylene (20) sorbitan monostearate, polyoxyethylene (20) sorbitan mono-oleate, polyoxypropylene/polyoxyethylene block copolymers, polyoxypropylene/polyoxyethylene/ethylenediamine block copolymers, ethoxylated castor oil and the like, where the proportion of surface-active agents, if present, can be about 0.0001 to 1% by weight, in particular about 0.001 to 0.1% by weight, based on the total formulation. Other suitable surfactant/emulsifying agents would be known to one of skill in the art and are listed in the CTFA International Cosmetic Ingredient Dictionary and Handbook, Vol. 2, 7th Edition (1997).

Other suitable aqueous vehicles include, but are not limited to, Ringer's solution and isotonic sodium chloride. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

Suitable petroleum hydrocarbons, i.e. mineral oils, paraffins and waxes from petroleum according to the present invention are: hard paraffin, liquid paraffin (Liquid Petrolatum or Paraffinum Liquidum), light liquid paraffin (Light Liquid Petrolatum or Paraffinum Perliquidium), white soft paraffin (White Petrolatum), yellow soft paraffin (Yellow Petrolatum), macrocrystalline paraffin waxes (which are mixtures which consist mainly of saturated C18-C30 hydrocarbons and smaller amounts of iso-alkanes and cycloalkanes with a molecular weight comprised between 250 and 450 g/mol and, although they are solids at room temperature, they have low melting points, usually comprised between 40° C. and 60° C.), microcrystalline paraffins waxes (which consist of C40-055 compounds which contain, in addition to normal hydrocarbons, large amounts of iso-alkanes and naphthenes with long alkyl side-chains, the iso-alkanes forming microcrystals, the microcrystalline paraffin waxes having mean molecular weights comprised between 500 and 800 g/mol, being solids at room temperature, and having melting points comprised between 60° C. and 90° C.), or mixtures thereof. Exemplary petroleum hydrocarbons are hard paraffin, liquid paraffin, light liquid paraffin, white soft paraffin or mixture thereof, being, for example, liquid paraffin, white soft paraffin or mixtures thereof.

Suitable fatty acids according to the present invention are C8-C24 carboxylic acids, saturated or unsaturated, purified or synthetic, from vegetable and animal fats and oils, such as 2-ethylhexanoic acid, arachidic acid, arachidonic acid, behenic acid, capric acid, caproic acid, caprylic acid, castor oil acid, coconut acid, corn acid, cottonseed acid, elaidic acid, erucic acid, gadoleic acid, isostearic acid, lauric acid, linoleic acid, linolenic acid, linseed acid, myristic acid, oleic acid, olein, olive acid, olive pomace, palm acid, palm kernel acid, palmitic acid (cetylic acid), palmitoleic acid, peanut acid, pelargonic acid, petroselinic acid, rapeseed acid, rice bran acid, ricinoleic acid, safflower acid, soy acid, stearic acid, sunflower seed acid, tall oil acid, tallow acid, undecanoic acid, undecylenic acid, wheat germ acid or mixtures thereof.

Fatty acid esters as used herein represent the covalent compounds formed between the fatty acids as described above and any suitable alcohol. Suitable fatty acid esters according to the present invention can be selected from (i) fats and oils, (ii) alkyl fatty esters, (iii) alkoxylated fatty acid esters and (iv) sorbitan fatty acid esters—(i) Fats and oils are the glyceryl esters of fatty acids (triglycerides) normally found in animal and plant tissues, Including those which have been hydrogenated to reduce or eliminate unsaturation. Also included are synthetically-prepared esters of glycerin and fatty acids (mono-, di-, and triglycerides). The fatty acids esterifying the different positions of glycerin can be different, giving rise to a large amount of possible combinations, including positional combinations. The position of the different fatty acids in natural triglycerides is not random, but rather It depends on the origin of the fat. The triglycerides more simple are those constituted by a sole fatty acid. Glyceryl esters of fatty acids can be advantageously chosen, for example, from the group consisting of synthetic, semi-synthetic and natural oils, as for example, animal fats and oils such as cow tallow, pig lard, bone oil, aquatic animal fats and oils (fish, such as herring, cod or sardine; cetaceans; etc.); and vegetable fats and oils such as avocado oil, almond oil, hazelnut oil, babassu palm oil, borage oil, peanut oil, canola oil, hemp oil, milk thistle oil, safflower oil, chufa oil, coconut oil, rapeseed oil, black cumin oil, wheat germ oil, sunflower oil, linseed oil, macadamia nut oil, corn oil, walnut oil, olive oil and Its byproducts such as olive pomace oil, palm oil and its fractions such as palm olein and palm stearin, evening primrose oil, rosehip oil, castor oil, rice bran oil, apricot kernel oil, cottonseed oil, pumpkinseed oil, palm kernel oil and its fractions such as palm kernel olein and palm kernel stearin, grape seed oil, sesame oil, soy oil, cocoa butter, shea butter and the like. Other examples of glyceryl esters are glyceryl monobehenate, glyceryl dibehenate, glyceryl monooleate, glyceryl dioleate, glyceryl monostearate, glyceryl distearate, glyceryl monopalmitosteareate and glyceryl dipalmitosteareate. (ii) Alkyl fatty esters represent esters of fatty acids as described above and linear or branched, saturated or unsaturated C1-C30 alcohols, ethylene glycol or propylene glycol. These fatty acid esters can be advantageously selected from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl laurate, 2-ethylhexyl palmitate. 2-ethylhexyl cocoate, 2-hexyldecyl stearate, 2-ethylhexyl isostearate, 2-octyldodecyl palmitate, cetyl palmitate, oleyl oleate, oleyl erucate, erucyl oleate, erucyl erucate, propyleneglycol monocaprylate, propyleneglycol dicaprylate, propyleneglycol monopalmitostearate, propyleneglycol monostearate, propyleneglycol alginate, ethyleneglycol monopalmitostearate, ethyleneglycol monostearate, as well as synthetic, semisynthetic and natural mixtures of such esters, such as jojoba oil (a natural mixture of esters of monounsaturated monocarboxylic acids with a C18-C24 chain with also monounsaturated monoalcohols and with a long C10-C24 chain).

Alkoxylated fatty acid esters are formed when a fatty acid is reacted with an alkylene oxide or with a preformed polymeric ether. The resulting product may be a monoester or a diester or a mixture of the two, depending on the reaction conditions. Typical representatives include PEG-6 isosteareate, PEG-2 stearate, PEG-4 stearate, PEG-8 stearate, PEG-12 stearate, PEG-20 stearate, PEG-40 stearate, PEG-50 stearate, PEG-100 stearate and PPG-17 dioleate. (iv) Sorbitan fatty acid esters are the reaction product of the esterification of sorbitan and one or more fatty acids as defined above. Examples of suitable sorbitan fatty acid esters include sorbitan monolaurate, sorbitan monooleate, sorbitan monopalmltate, sorbitan monostearate, sorbitan sesquioleate, sorbitan trioleate and sorbitan tristearate, and polysorbates such as polysorbate 20, polysorbate 40, polysorbate 60, polysorbate 65, polysorbate 80 or polysorbate 85.

Suitable fatty alcohols according to the present invention are C6-C24 alcohols from vegetable and animal fats and oils, such as 2-octyldodecanol, 2-ethylhexanoyl alcohol, arachidyl alcohol, behenyl alcohol, caprylic alcohol, caproyl alcohol, capric alcohol, castor oil alcohol, ceterayl alcohol, palmityl (cetyl) alcohol, coconut alcohol, cotton alcohol, decyl alcohol, elaidyl alcohol, erucyl alcohol, gadoleyl alcohol, isostearyl alcohol, lauryl alcohol, linoleyl alcohol, linseed alcohol, myristyl alcohol, olein alcohol, olive pomace alcohol, oleyl alcohol, olive alcohol, palm alcohol, palm kernel alcohol, palmitoyl alcohol, petroselinic alcohol, rapeseed alcohol, ricinoleyl alcohol, safflower alcohol, soy alcohol, stearyl alcohol, sunflower alcohol, tall oil alcohol, tallow alcohol, tridecyl alcohol, or technical grade mixtures thereof such as cetostearyl alcohol.

Suitable fatty alcohol ethers according to the present invention are ethers formed from the reaction of a fatty alcohol as defined above with an alkylene oxide, generally ethlyene oxide or propylene oxide. Fatty alcohols ethers can be advantageously selected from the group consisting of ceteth-20, isosteareth-10, myreth-10, laureth-16, oleth-16, polyoxyl 6 cetostearyl ether, polyoxyl 20 catostearyl ether, polyoxyl 25 cetostearyl ether, polyoxyl 2 cetyl ether, polyoxyl 10 cetyl ether, polyoxyl 20 cetyl ether, polyoxyl 4 lauryl ether, polyoxyl 9 lauryl ether, polyoxyl 23 lauryl ether, polyoxyl 2 oleyl ether, polyoxyl 10 oleyl ether, polyoxyl 20 oleyl ether, polyoxyl 2 stearyl ether, polyoxyl 10 stearyl ether, polyoxyl 21 stearyl ether or polyoxyl 100 stearyl ether.

Suitable fatty alcohol esters are the product of the reaction of a fatty alcohol as defined above and a linear of branched, saturated or unsaturated C1-C5 carboxylic acid. Examples of fatty alcohol esters are lauryl acetate, myristyl acetate, cetyl acetate, stearyl acetate and stearyl propionate.

Suitable aromatic alcohols according to the present invention can be selected from (i) arylalkanols, (ii) aryloxyalkanols (glycol monoaryl ethers) and (iii) oligoalkanol aryl ethers. The (i) arylalkanols used according to the Invention have the formula Ar—(CHR)n-OH where R independently represents H or C1-C6 alkyl, with n being an integer, for example, between 1 to 10, or, for example 1 to 6, or 1, 2, 3 or 4. The group Ar can be a substituted or unsubstituted aryl group, for example phenyl or naphtyl. Example arylalkanols are benzyl alcohol, 3-phenylpropan-1-ol, phenethyl alcohol, veratryl alcohol (3,4-dimethoxyphenylmethyl alcohol) and 2-methyl-1-phenyl-2-propanol. The (ii) aryloxyalkanols used according to the invention have the formula Ar—O—(CHR)n-OH where R independently represents H or C1-C6 alkyl, with n being an integer, for example, between 2 to 10, or 2 to 6, or 2 or 3. The group Ar can be a substituted or unsubstituted aryl group, for example phenyl or naphtyl. Example arlyoxyalkanols used according to the invention are phenoxyethanol, 1-phenoxypropan-2-ol, 2-phenoxylpropan-1-ol, 3-phenoxypropan-1-ol, or mixtures thereof. The (iii) oligoalkanol aryl ethers include, for example, phenoxy diethanol, triethanol and oligoethanol, and phenoxy dipropanol, tripropanol and oligopropanol.

Suitable polyols according to the present invention include water-soluble polyols such as polyhydric alcohols with two or more hydroxyl groups in their molecule. Specific examples can include ethylene glycol, propylene glycol, 1,3-butylene glycol, 1,4-butylene glycol, hexylene glycol, dipropylene glycol, glucose, fructose, galactose, mannose, ribose, erythrose, maltose, maltitose, maltotriose, sucrose, xylitol, sorbitol, threitol, erythritol, glycerol, polyglycerol and starch alcohols. Exemplary polyols are ethylene glycol, propylene glycol, 1,3-butylene glycol, 1,4-butylene glycol, dipropylene glycol, hexylene glycol, glycerol, polyglycerol, and mixtures thereof.

Suitable alkyleneglycol esters are esters of ethylene glycol or propylene glycol with the C6-C24 fatty acids defined above. Alkyleneglycol esters can be advantageously selected from the group consisting of ethylene glycol monopalmitostearate, ethylene glycol monostearate, propylene glycol monocaprylate, propylene glycol diceprylate, propylene glycol dicaprylocaprate, propylene glycol monopalmitostearate, propylene glycol monostearate or propylene glycol alginate.

Suitable alkylene glycol ethers are polymers of ethylene oxide (polyethylene glycol monomethyl ether) or propylene oxide (polypropylene glycol monomethyl ether). Alkyleneglycol ethers can be advantageously selected from the group consisting of PEG 200, PEG 400, PEG 540, PEG 600, PEG 900, PEG 1000, PEG 1450, PEG 1540, PEG 2000, PEG 3000, PEG 3350, PEG 4000, PEG 4600, PEG 8000, PPG-9, PPG-10, PPG-17, PPG-20, PPG-26, PPG-30 or PPG-55.

Suitable natural waxes according to the present invention are the candelilla wax, carnauba wax, Japan wax, esparto wax, cork wax, guaruma wax, rice wax, sugar cane wax, ouricury wax, montan wax, beeswax, shellac wax, espermaceti, wool lanolin (wax), uropygial fat wax, ceresin waxes, peat waxes, ozokerite, as well as chemically modified waxes (hard waxes) for example, montan wax esters, waxes obtained by the Fischer-Tropsch process, hydrogenated jojoba waxes and synthetic waxes.

Silicones suitable according to the present invention are cyclic and/or linear silicones, which can be found as monomers generally characterized by structural elements such as: where the silicon atoms can be substituted by alkyl or aryl radicals equal or different, represented here generally by R1-R4 groups.

Linear silicones with siloxane units suitable according to the present invention are generally characterized by structural elements such as: where the silicon atoms can be substituted by alkyl or aryl radicals equal or different, are represented here in general by R1-R4 groups (meaning the number of different radicals is not necessarily limited to 4), m can take values from 2 to 200.000.

Cyclic silicones suitable according to the present invention are generally characterized by structural elements such as: where the silicon atoms can be substituted by alkyl or aryl radicals equal or different, represented here generally by R1-R4 groups (meaning the number of different radicals is not necessarily limited to 4), n can take values of 3/2 to 20. Fractional values of n indicate that it may be odd numbers of siloxane groups present in the ring. Specific examples include a cyclic methyl siloxane having the formula [(CH3)2SiO]x in which x is 3-6, or short chain linear methyl siloxanes having the formula ((CH3)2SiO[(CH3)2SiO]ySi(CH3)3 in which y is 0-5.

Some suitable cyclic methyl siloxanes are hexamethylcyclotrisiloxanes (D3), a solid with a boiling point of 134° C. and the formula [(Me2)SiO]3; octamethylcyclotetrasiloxane (D4) with a boiling point of 176° C., a viscosity of 2.3 mm2/s, and the formula [(Me2)SiO]4; decamethylcyclopentasiloxane (D5) (cyclomethicone) with a boiling point of 210° C., a viscosity of 3.87 mm2/s, and the formula [(Me2)SiO]5; and dodecamethylcyclohexasiloxane (D6) with a boiling point of 245° C., a viscosity of 6.62 mm2/s and the formula [(Me2)siO]6.

Some suitable short linear methyl siloxane are hexamethyldisiloxane (MM) with a boiling point of 100° C., viscosity of 0-65 mm2/s, and formula Me3SiOMe3; octamethyltrisiloxane (MDM) with a boiling point of 152° C., viscosity of 1.04 mm2/s, and formula MesSiOMe2SiOSiMe3; decamethyltetrasiloxane (MD2M) with a boiling point of 194° C., viscosity of 1.53 mm2/s, and formula Me3SiO(MeSiO)2SiMe3; dodecamethylpentasiloxane (MD3M) with a boiling point of 229° C., viscosity of 2.06 mm2/s, and formula Me3SiO(Me2SiO)3SiMe3; tetradecamethylhexasiloxane (MD4M) with a boiling point of 245° C., viscosity of 2.63 mm2/s, and formula Me3SiO(Me2SiO)4SiMe3; and hexadecamethylheptasiloxane (MD5M) with a boiling point of 270° C., viscosity of 3.24 mm2/s, and formula Me3SiO(Me2SiO)5SiMe3.

Furthermore, long chain linear siloxanes such as phenyltrimethicone, bis(phenylpropyl)dimethicone, dimethicone, dimethiconol, cyclomethicone (octametilciclotetrasiloxane), hexamethylcyclotrisiloxane, poly(dimethylsiloxane), cetyldimothicone and behenoxy dimethicone are also included.

In addition, mixtures of cyclomethicone and isotridecyl isononanoate and of cyclomethicone and 2-ethylhexyl isostearate are also suitable silicones according to the invention.

Although not crucial, the dilution and/or formulation of the active ingredients of the compositions described herein, in a physiologically acceptable topical vehicle, can be important and useful in providing the final dosage concentration. The compositions can be supplied in solid, semi-solid or liquid forms, including tablets, capsules, powders, liquids, and suspensions. The compositions described herein can therefore encompass concentrated forms for subsequent dilution before use or sale. The compositions can comprise any physiologically acceptable topical excipients including, but not limited to, gels, lotions, creams, ointments, and liquids, as further elaborated herein. The topical pharmaceutical composition according to the invention can be formulated in the form of a cream, a gel, an oleogel, an ointment, a paste, a suspension, a lotion, a foam, a spray, an aerosol or a solution. In one embodiment, the invention can be in the form of a cream, an ointment or a lotion, shampoo or soap, other topical or dermatological vehicle.

The Food and Drug Administration (FDA), Center for Drug Evaluation and Research (CDER) Data Standards Manual, Dosage Form (version 08) defines ointment as “a semisolid dosage form, usually containing less than 20% water and volatiles and more than 50% hydrocarbons, waxes, or polyols as the vehicle, which is generally for external application to the skin or mucous membranes”.

The Food and Drug Administration (FDA), Center for Drug Evaluation and Research (CDER) Data Standards Manual, Dosage Form (version 08) defines cream as “an emulsion, semisolid dosage form, usually containing more than 20% water and volatiles and/or less than 50% hydrocarbons, waxes, or polyols as the vehicle, which is generally for external application to the skin or mucous membranes”.

The topical pharmaceutical compositions according to the invention may optionally further comprise other well-known pharmaceutically and/or cosmetically acceptable additives, such as, e.g, anti-irritants, antioxidants, buffering agents (pH adjusting agents), chelating agents, emollients, penetration enhancing agents, preservative agents, solubilizing agents, thickening agents, wetting agents, and the like, or mixtures thereof.

Examples of suitable anti-irritants are aloe vera, chamomile, alpha-bisabolol, cola nitida extract, green tea extract, tea tree oil, licoric extract, batyl alcohol (a-octadecyl glyceryl ether), selachyl alcohol (α-9-octadecenyl glyceryl ether), chimyl alcohol (α-hexadecyl glyceryl ether), panthenol, allantoin, caffeine or other xanthines, glycyrrhizic acid and derivatives thereof, and mixtures thereof.

Antioxidants used can be any antioxidants which are suitable or customary for cosmetic and/or dermatological applications. Suitable antioxidants can include ones from the group consisting of amino acids (for example glycine, histidine, tyrosine, tryptophan) and derivatives thereof, imidazoles (e.g. urocanic acid) and derivatives thereof, peptides such as D,L-carnosine, D-carnosine, L-carnosine and derivatives thereof (e.g. anserine), carotenoids, carotenes (e.g. a-carotene, (3-carotene, lycopene) and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, y-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) and sulphoximine compounds (e.g. buthionine sulphoximines, homocysteine sulphoximine, buthionine sulphones, penta-, hexa-, heptathionine sulphoximine) in very small tolerated doses (e.g. pmol to mol/kg), also (metal) chelating agents (e.g. a-hydroxy fatty acids, palmitic acid, phytic acid, lactoferrin), a-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. y-linolenic acid, linoleic acid, oleic acid), folic acid and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), and coniferylbenzoate of benzoin, rutinic acid and derivatives thereof, ferulic acid and derivatives thereof, butylated hydroxytoluene, butylated hydroxyanisole, nordihydroguaiac resin acid, nordihydroguaiaretic acid, trihydroxybutyrophenone, uric acid and derivatives thereof, mannose and derivatives thereof, zinc and derivatives thereof (e.g. ZnO, ZnSO4), selenium and derivatives thereof (e.g. selenium methionine), stilbenes and derivatives thereof (e.g. stilbene oxide, trans-stilbene oxide) and the derivatives (salts, esters, ethers, sugars, nucleotides, nucleosides, peptides and lipids) of said active ingredients which are suitable according to the invention.

Any pharmaceutically acceptable buffering agents to adjust the pH of the topical pharmaceutical compositions according to the invention to be within the acceptable range for topical administration, for example, in the range of 3.0 to 6.0, or in the range of 3.5 to 5.0, can be used. For example the inclusion in the composition of a pharmaceutically acceptable acid such as acetic, citric, fumaric, phosphoric, hydrochloric, lactic or nitric acids or the like, or a mixture thereof. It will also be understood that certain compositions of the invention can have a pH in the desired range without inclusion of a pH adjusting agent specifically for that purpose. Typically, however, an acidic buffer system is present in the composition to achieve the desired pH. An acidic buffer system comprises an acidulant and a buffering agent. Suitable acidulants will be known to those of skill in the art and illustratively include acetic, citric, fumaric, hydrochloric, phosphoric, lactic and nitric acids and the like, and mixtures thereof. Suitable buffering agents will likewise be known to those of skill in the art and illustratively include potassium metaphosphate, potassium phosphate, sodium phosphate, sodium acetate, sodium citrate and the like, and mixtures thereof.

Suitable emollients, which can be used in the composition of the present invention include, for example, dodecane, squalane, cholesterol, isohexadecane, isononyl isononanoate, PPG ethers, petrolatum, lanolin, safflower oil, castor oil, coconut oil, cottonseed oil, palm kernel oil, palm oil, peanut oil, soybean oil, polyol carboxylic acid esters, derivatives thereof, and the like, and combinations thereof.

In one aspect, the pharmaceutical composition for topical administration or transdermal administration of a muscarinic acetylcholine receptor antagonist(s) may comprise one or more penetration enhancing agent or co-solvent for transdermal or topical delivery. A penetration enhancer is an excipient that aids in the diffusion of the active through the stratum corneum. Many penetration enhancers also function as co-solvents that are thought to increase the thermodynamic activity or solubility of the muscarinic acetylcholine receptor antagonist in the composition. Penetration enhancers are also known as accelerants, adjuvants or sorption promoters. Examples of suitable penetration enhancing agents can Include, e.g., dimethylsulfoxide (DMSO), N-methyl pyrrolidone, dimethyl formamide (DMF), allantoin, urazole, N,N-dimethylacetamide (DMA), decylmethylsulfoxide, polyethylene glycol monolaurate, propylene glycol, propylene glycol monolaurate, glycerol monolaurate, lecithin, the 1-substituted azacycloheptan-2-ones, particularly 1-n-dodecylcyclazacycloheptan-2-one, alcohols, glycerin, hyaluronic acid, transcutol, and the like, and combinations thereof. Certain oil components (e.g., certain vegetable oils such as, e.g., safflower oil, cottonseed oil and corn oil) also can exhibit penetration enhancing properties.

Another class of penetration enhancers are alkanones, such as N-heptane, N-octane, N-nonane, N-decane, N-undecane, N-dodecane, N-tridecane, N-tetradecane and N-hexadecane. Alkanones are thought to enhance the penetration of an active agent by altering the stratum corneum. A further class of penetration enhancers are alkanol alcohols, such as ethanol, propanol, butanol, 2-butanol, pentanol, 2-pentanol, hexanol, octanol, nonanol, decanol and benzyl alcohol. Low molecular weight alkanol alcohols, i.e., those with 6 or less carbons, may enhance penetration in part by acting as solubilizing agents, while more hydrophobic alcohols may increase diffusion by extracting lipids from the stratum corneum. A further class of penetration enhancers are fatty alcohols, such as oleyl alcohol, caprylic alcohol, decyl alcohol, lauryl alcohol, 2-lauryl alcohol, myristyl alcohol, cetyl alcohol, stearyl alcohol, oleyl alcohol, linoleyl alcohol and linolenyl alcohol. Polyols, including propylene glycol, polyethylene glycol, ethylene glycol, diethylene glycol, triethylene glycol, dipropylene glycol, glycerol, propanediol, butanediol, pentanediol, hexanetriol, propylene glycol monolaurate and diethylene glycol monomethyl ether (transcutol), can also enhance penetration. Some polyols, such as propylene glycol, may function as a penetration enhancer by solvating alpha-kertin and occupying hydrogen bonding sites, thereby reducing the amount of active-tissue binding.

Another class of penetration enhancers are amides, including urea, dimethylacetamide, diethyltoluamide, dimethylformamide, dimethyloctamide, dimethyldecamide and biodegradable cyclic urea (e.g., 1-alkyl-4-imidazolin-2-one). Amides have various mechanisms of enhancing penetration. For example, some amides, such as urea increase the hydration of the stratum corneum, act as a keratolytic and create hydrophilic diffusion channels. In contrast, other amides, such as dimethylacetamide and dimethylformamide, increase the partition to keratin at low concentrations, while increasing lipid fluidity and disrupting lipid packaging at higher concentrations. Another class of penetration enhancing agents are pyrrolidone derivatives, such as 1-methyl-2-pyrrolidone, 2-pyrrolidone, 1-lauryl-2-pyrrolidone, 1-methyl-4-carboxy-2-pyrrolidone, 1-hexyl-4-carboxy-2-pyrrolidone, 1-lauryl-4-carboxy-2-pyrrolidone, 1-methyl-4-methoxycarbonyl-2-pyrrolidone, 1-hexyl-4-methoxycarbonyl-2-pyrrolidone, 1-lauryl-4-methoxycarbonyl-2-pyrrolidone, N-methyl-pyrrolidone, N-cyclohexylpyrrolidone, N-dimethylaminopropyl-pyrrolidone, N-cocoalkypyrrolidone and N-tallowalkypyrrolidone, as well as biodegradable pyrrolidone derivatives, including fatty acid esters of N-(2-hydroxyethyl)-2-pyrrolidone. In part, pyrrolidone derivatives enhance penetration through interactions with the keratin in the stratum corneum and lipids in the skin structure. An additional class of penetration enhancers are cyclic amides, including 1-dodecylazacycloheptane-2-one also known as Azone® (Azone is a registered trademark of Echo Therapuetics Inc., Franklin, Mass., USA), 1-geranylazacycloheptan-2-one, 1-farnesylazacycloheptan-2-one, 1-geranylgeranylazacycloheptan-2-one, 1-(3,7-dimethyloctyl)-azacycloheptan-2-one, 1-(3,7,11-trimethyldodecyl)azacyclohaptan-2-one, 1-geranylazacyclohexane-2-one, 1-geranylazacyclopentan-2,5-dione and 1-farnesylazacyclopentan-2-one. Cyclic amides, such as Azone®, enhance the penetration of active agents in part by affecting the stratum corneum's lipid structure, increasing partitioning and increasing membrane fluidity. Additional classes of penetration enhancers include diethanolamine, triethanolamine and hexamethylenlauramide and its derivatives.

Additional penetration enhancers include linear fatty acids, such as octanoic acid, linoleic acid, valeric acid, heptanoic acid, pelagonic acid, caproic acid, capric acid, lauric acid, myristric acid, stearic acid, oleic acid and caprylic acid. Linear fatty acids enhance penetration in part via selective perturbation of the intercellular lipid bilayers. In addition, some linear fatty acids, such as oleic acid, enhance penetration by decreasing the phase transition temperatures of the lipid, thereby increasing motional freedom or fluidity of the lipids. Branched fatty acids, including isovaleric acid, neopentanoic acid, neoheptanoic acid, nonanoic acid, trimethyl hexaonic acid, neodecanoic acid and isostearic acid, are a further class of penetration enhancers. An additional class of penetration enhancers are aliphatic fatty acid esters, such as ethyl oleate, isopropyl n-butyrate, isopropyl n-hexanoate, isopropyl n-decanoate, isopropyl myristate (“IPM”), isopropyl palmitate and octyldodecyl myristate. Aliphatic fatty acid esters enhance penetration by increasing diffusivity in the stratum corneum and/or the partition coefficient. In addition, certain aliphatic fatty acid esters, such as IPM, enhance penetration by directly acting on the stratum corneum and permeating into the liposome bilayers thereby increasing fluidity. Alkyl fatty acid esters, such as ethyl acetate, butyl acetate, methyl acetate, methyl valerate, methyl propionate, diethyl sebacate, ethyl oleate, butyl stearate and methyl laurate, can act as penetration enhancers. Alkyl fatty acid esters enhance penetration in part by increasing the lipid fluidity.

Other penetration enhancers are bile salts, such as sodium cholate, sodium salts of taurocholic acid, glycolic acids and desoxycholic acids. Lecithin also has been found to have penetration enhancing characteristics. An additional class of penetration enhancers are terpenes, which include hydrocarbons, such as d-limonene, alpha-pinene and beta-carene; alcohols, such as, alpha-terpineol, terpinen-4-ol and carvol; ketones, such ascarvone, pulegone, piperitone and menthone; oxides, such as cyclohexene oxide, limonene oxide, alpha-pinene oxide, cyclopentene oxide and 1,8-cineole; and oils such as ylang ylang, anise, chenopodium and eucalyptus. Terpenes enhance penetration in part by disrupting the intercellular lipid bilayer to increase diffusivity of the active and opening polar pathways within and across the stratum corneum. Organic acids, such as salicylic acid and salicylates (including their methyl, ethyl and propyl glycol derivates), citric acid and succinic acid, are penetration enhancers. Another class of penetration enhancers are cyclodextrins, including 2-hydroxypropyl-beta-cyclodextrin and 2,6-dimethyl-beta-cyclodextrin. Cyclodextrins enhance the permeation of active agents by forming inclusion complexes with lipophilic actives and increasing their solubility in aqueous solutions.

For a discussion of use of penetration enhancers in topical formulations see generally, Percutaneous Penetration Enhancers (Eric W. Smith & Howard I. Maibach eds. 1995); Ghosh, T. K. et al. 17 Pharm. Tech. 72 (1993); Ghosh, T. K. et al. 17 Pharm. Tech. 62 (1993); Ghosh, T. K. et al. 17 Pharm. Tech. 68 (1993), all of which citations are hereby incorporated herein by reference. The penetration enhancer should be pharmacologically inert, non-toxic, and non-allergenic, have rapid and reversible onset of action, and be compatible with the compositions of the invention.

Examples of suitable preservatives to prevent microbial contamination are alkylparabens, particularly methylparaben, propylparaben and butylparaben; sodium benzoate; butylated hydroxy toluene; butylated hydroxyanisole; ethylenediamine tetraacetic acid; chlorobutanol; benzyl alcohol; phenylethylalcohol; dehydroacetic acid; sorbic acid; potassium sorbate; benzalkonium chloride; benzethonium chloride; and mixtures thereof. The amount of preservative generally utilized will vary depending upon the preservative selected.

Examples of solubilizing agents are, for example, nonionic surfactants from at least one of the following groups: products of the addition of 1 to 30 moles of ethylene oxide and/or 0 to 5 moles of propylene oxide onto linear C8-C22 fatty alcohols, C12-C22 fatty acids and alkyl phenols containing 8 to 15 carbons in the alkyl group; alkyl and/or alkenyl oligoglycosides containing 8 to 22 carbons in the alkyl group and ethoxylated analogs thereof; addition products of 1 to 15 moles of ethylene oxide with castor oil and/or hydrogenated castor oil; addition products of 15 to 60 moles of ethylene oxide with castor oil and/or hydrogenated castor oil; partial esters of glycerol and/or sorbitan with unsaturated or saturated, linear or branched fatty acids containing 12 to 22 carbons and/or hydroxycarboxylic acids containing 3 to 18 carbon atoms and addition products thereof with 1 to 30 moles of ethylene oxide; mixtures of alkoxylated glycerides and alkoxylated glycerin, partial esters of polyglycerol (average degree of self condensation 2 to 8), polyethylene glycol (weight average molecular weight 400 to 5000), trimethylolpropane, pentaerythritol, sugar alcohols (for example sorbitol), alkyl glucosides (for example methyl glucoside, butyl glucoside, lauryl glucoside) and polyglucosides (for example cellulose) with saturated and/or unsaturated, linear or branched fatty acids containing 12 to 22 carbons and/or hydroxycarboxylic acids containing 3 to 18 carbons and addition products thereof with 1 to 30 moles of ethylene oxide; mixed esters of pentaerythritol, fatty acids; citric acid and fatty alcohol and/or mixed esters of fatty acids containing 6 to 22 carbons, methyl glucose and polyols, for example, glycerol or polyglycerol; mono-, di- and trialkyl phosphates and mono-, di- and/or tri-PEG-alkyl phosphates and salts thereof; block copolymers, for example Polyethyleneglycol-30 Dipolyhydroxystearate; polymer emulsifiers; polyalkylene glycols and alkyl glyceryl ethers. Exemplary solubilizing agents are products of the addition of 1 to 30 moles of ethylene oxide and/or 0 to 5 moles of propylene oxide onto linear C6-C22 fatty alcohols such as lauryl, myristyl, cetyl (palmityl), stearyl, oleyl, and ricinoleyl alcohols, or technical grade mixtures thereof such as cetostearyl alcohol or palmitoleyl alcohol.

A thickening agent or viscosity-enhancing agent can be included to generally thicken the liquid pharmaceutical compositions. While any suitable thickening agent can be included in the compositions of the present invention, a example thickening agent, when used, includes one or more of acacia, alginic acid bentonite, carbomer, carboxymethylcellulose calcium or sodium, cetostearyl alcohol, methyl cellulose, ethylcellulose, glycerin, gelatin guar gum, hydroxyethylcellulose, hydroxypropylcellulose, hydroxypropylmethylcellulose, maltodextrin, polyvinyl alcohol, povidone, propylene carbonate, propylene glycol alginate, sodium alginate, sodium starch glycolate, starch tragacanth, and xanthan gum, and any combination thereof. In one particular embodiment, the thickening agents are glycerin, hydroxypropylmethylcellulose, and xanthan gum, and any combination thereof.

Examples of wetting agents (chemical substances that increase the spreading and penetrating properties of a liquid by lowering its surface tension) include one or more cationic surfactants, such as benzalkonium chloride; non-ionic surfactants such as polyoxyethylene and polyoxypropylene block copolymers; polyoxyethylene fatty acid glycerides and oils (such as polyoxyethylene (6) caprylic/capric mono- and diglycerides), polyoxyethylene (40) hydrogenated castor oil; polyoxyethylene sorbitan esters, such as polysorbate 20 and polysorbate 80; propylene glycol fatty acid esters, such as propylene glycol laureate; glyceryl fatty acid esters, such as glyceryl monostearate; sorbitan esters, such as sorbitan monolaurate, sorbitan monooleate, sorbitan monopalmitate and sorbitan monostearate; glyceryl fatty acid esters, for example glyceryl monostearate; anionic surfactants such as sodium lauryl sulphate, sodium lauryl ether sulphate; or fatty acids and salts thereof, such as oleic acid, sodium oleate and triethanolemine oleate.

The viscosity of the topical pharmaceutical composition of the invention will depend on the form of the composition. For instance, in the case of a cream, the viscosity is typically in the range of 2,000 to 15,000 mPa·s, or for example, in the range of 2,500 to 10,000 mPa·s, or in the range of 3,000 to 7,000 mPa·s measured at 20° C. using a DIN-Rotations Rheometer (Paar Physica); Measuring System Z 3 DIN; D=57 l/s.

In the case of a gel, the viscosity is typically in the range of 300 to 1,500 mPa·s, for example in the range of 500 to 1,200 mPa·s, or in the range of 600 to 900 mPa·s measured at 20° C. using a DIN-Rotations Rheometer (Paar Physica); Measuring System Z 3 DIN; D=57.2/s.

The pharmaceutical compositions may further include one or more inert excipients, which include water, buffered aqueous solutions, surfactants, volatile liquids, starches, polyols, granulating agents, microcrystalline cellulose, diluents, lubricants, acids, bases, salts, emulsions, such as oil/water emulsions, oils such as mineral oil and vegetable oil, wetting agents, chelating agents, antioxidants, sterile solutions, complexing agents, disintegrating agents and the like. The CTFA Cosmetic Ingredient Handbook, Seventh Edition, 1997 and the Eighth Edition, 2000, which is incorporated by reference herein in its entirety, describes a wide variety of cosmetic and pharmaceutical ingredients commonly used in skin care compositions, which are suitable for use in the compositions of the present invention. Examples of these functional classes disclosed in this reference include: absorbents, abrasives, anticaking agents, antifoaming agents, antimicrobial agents, antioxidants, binders, biological additives, buffering agents, bulking agents, chelating agents, chemical additives, colorants, cosmetic astringents, cosmetic biocides, denaturants, drug astringents, external analgesics, film formers, fragrance components, humectants, opacifying agents, pH adjusters, plasticizers, preservatives, reducing agents, skin bleaching agents, skin-conditioning agents (emollient, humectants, miscellaneous, and occlusive), skin protectants, solvents, foam boosters, hydrotropes, solubilizing agents, steroidal anti-inflammatory agents, surfactants/emulsifying agents, suspending agents (nonsurfactant), sunscreen agents, topical analgesics, ultraviolet light absorbers, SPF boosters, thickening agents, waterproofing agents, and viscosity increasing agents (aqueous and nonaqueous).

Chelating agents which can be used to form pharmaceutical compositions and dosage forms of the invention include, but are not limited to, ethylene diaminetetraacetic acid (EDTA), EDTA disodium, calcium disodium edetate, EDTA trisodium, albumin, transferrin, desferoxamine, desferal, desferoxamine mesylate, EDTA tetrasodium and EDTA dipotassium, sodium metasilicate or combinations of any of these. In some embodiments, up to about 0.1% W/V of a chelating agent, such as EDTA or its salts, is added to the formulations of the invention.

Preservatives which can be used to form pharmaceutical compositions and dosage forms of the invention include, but are not limited to, purite, peroxides, perborates, imidazolidinyl urea, diazolidinyl urea, phenoxyethanol, alkonium chlorides including benzalkonium chlorides, methylparaben, ethylparaben and propylparaben. In other embodiments, suitable preservatives for the compositions of the invention include: benzalkonium chloride, purite, peroxides, perborates, thimerosal, chlorobutanol, methyl paraben, propyl paraben, phenylethyl alcohol, edetate disodium, sorbic acid, Onamer M, or other agents known to those skilled in the art. In some embodiments of the invention, such preservatives may be employed at a level of from 0.004% to 0.02% W/V. In some compositions of the present application the preservative, for example, benzalkonium chloride, methyl paraben, and/or propyl paraben, may be employed at a level of from about 0.001% to less than about 0.01%, e.g. from about 0.001% to about 0.008%, or about 0.005% W/V. It has been found that a concentration of benzalkonium chloride of about 0.005% may be sufficient to preserve the compositions of the present invention from microbial attack. One of skill in the art could determine the proper concentration of ingredients as well as combinations of various ingredients for generating a suitable topical formulation. For example, ophthalmic drops or formulations for application to skin may use a mixture of methyl and propyl parabens at about 0.02% W/V and about 0.04% W/V respectively. In some embodiments, these formulations use methyl paraben and/or propyl paraben in amounts up to about 0.02% W/V and up to about 0.04% W/V respectively, which encompasses the embodiments where no methyl paraben or no propyl paraben is used.

Lubricants which can be used to form pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, or mixtures thereof.

Thickening agents which can be used to form pharmaceutical compositions and dosage forms of the invention include, but are not limited to, isopropyl myristate, isopropyl palmitate, isodecyl neopentanoate, squalene, mineral oil, C12-C15 benzoate and hydrogenated polyisobutene. Those agents which would not disrupt other compounds of the final product, such as non-ionic thickening agents may be desirable. The selection of additional thickening agents is well within the skill of one in the art.

Skin conditioning agents can be emollients, humectants and moisturizers. A humectant is a moistening agent that promotes retention of water due to its hygroscopic properties. Suitable skin conditioning agents include urea; guanidine; aloe vera; glycolic acid and glycolate salts such as ammonium and quaternary alkyl ammonium; lactic acid and lactate salts such as sodium lactate, ammonium lactate and quaternary alkyl ammonium lactate; polyhydroxy alcohols such as sorbitol, glycerol, mannitol, xylitol, hexanetriol, propylene glycol, butylene glycol, hexylene glycol, polymeric glycols such as polyethylene glycol and polypropylene glycol; carbohydrates such as alkoxylated glucose; starches; starch derivatives; glycerin; pyrrolidone carboxylic acid (PCA); lactamide monoethanolamine; acetamide monoethanolamine; volatile silicone oils; nonvolatile silicone oils; and mixtures thereof. Suitable silicone oils can be polydialkylsiloxanes, polydiarylsiloxanes, polyalkarylsiloxanes and cyclomethicones having 3 to 9 silicon atoms.

An emollient is an oleaginous or oily substance which helps to smooth and soften the skin, and may also reduce its roughness, cracking or irritation. Typical suitable emollients include mineral oil having a viscosity in the range of 50 to 500 centipoise (cps), lanolin oil, coconut oil, cocoa butter, olive oil, almond oil, macadamia nut oil, aloe extracts such as aloe vera lipoquinone, synthetic jojoba oils, natural sonora jojoba oils, safflower oil, corn oil, liquid lanolin, cottonseed oil and peanut oil. In some embodiments, the emollient is a cocoglyceride, which is a mixture of mono, di and triglycerides of cocoa oil, sold under the trade name of Myritol 331 from Henkel KGaA, or Dicaprylyl Ether available under the trade name Cetiol OE from Henkel KGaA or a C12-C15 Alkyl Benzoate sold under the trade name Finsolv Tenn. from Finetex. Another suitable emollient is DC 200 Fluid 350, a silicone fluid, available from Dow Corning Corp.

Other suitable emollients include squalane, castor oil, polybutene, sweet almond oil, avocado oil, calophyllum oil, ricin oil, vitamin E acetate, olive oil, silicone oils such as dimethylopolysiloxane and cyclomethicone, linolenic alcohol, oleyl alcohol, the oil of cereal germs such as the oil of wheat germ, isopropyl palmitate, octyl palmitate, isopropyl myristate, hexadecyl stearate, butyl stearate, decyl oleate, acetyl glycerides, the octanoates and benzoates of (C12-C15) alcohols, the octanoates and decanoates of alcohols and polyalcohols such as those of glycol and glyceryl, ricinoleates esters such as isopropyl adipate, hexyl laurate and octyl dodecanoate, dicaprylyl maleate, hydrogenated vegetable oil, phenyltrimethicone, jojoba oil and aloe vera extract.

Other suitable emollients which are solids or semi-solids at ambient temperatures may be used. Such solid or semi-solid cosmetic emollients include glyceryl dilaurate, hydrogenated lanolin, hydroxylated lanolin, acetylated lanolin, petrolatum, isopropyl lanolate, butyl myristate, cetyl myristate, myristyl myristate, myristyl lactate, cetyl alcohol, isostearyl alcohol and isocetyl lanolate. One or more emollients can optionally be included in the formulation.

Anti-oxidants which can be used to form pharmaceutical compositions and dosage forms of the invention include, but are not limited to, propyl, octyl and dodecyl esters of gallic acid, butylated hydroxyanisole (BHA, usually purchased as a mixture of ortho and meta isomers), green tea extract, uric acid, cysteine, pyruvate, nordihydroguaiaretic acid, ascorbic acid, salts of ascorbic acid such as ascorbyl palmitate and sodium ascorbate, ascorbyl glucosamine, vitamin E (i.e., tocopherols such as a-tocopherol), derivatives of vitamin E (e.g., tocopheryl acetate), retinoids such as retinoic acid, retinol, trans-retinol, cis-retinol, mixtures of trans-retinol and cis-retinol, 3-dehydroretinol and derivatives of vitamin A (e.g., retinyl acetate, retinal and retinyl palmitate, also known as tetinyl palmitate), sodium citrate, sodium sulfite, lycopene, anthocyanids, bioflavinoids (e.g., hesperitin, naringen, rutin and quercetin), superoxide dismutase, glutathione peroxidase, butylated hydroxytoluene (BHT), indole-3-carbinol, pycnogenol, melatonin, sulforaphane, pregnenolone, lipoic acid and 4-hydroxy-5-methyl-3[2H]-furanone.

Skin protecting agents are agents that protect the skin against chemical irritants and/or physical irritants, e.g., UV light, including sunscreens, anti-acne additives, anti-wrinkle and anti-skin atrophy agents. Suitable sunscreens as skin protecting agents include 2-ethylhexyl p-methoxycinnamate, 2-ethylhexyl N,N-dimethyl-p-aminobenzoate, p-aminobenzoic acid, 2-phenylbenzimidazole-5-sulfonic acid, octocrylene, oxybenzone, homomethyl salicylate, octyl salicylate, 4,4 ‘-methoxy-t-butyldibenzoylmethane, 4-isopropy dibenzoylmethane, 3-benzylidene camphor, 3-(4-methylbenzylidene) camphor, anthanilates, ultrafine titanium dioxide, zinc oxide, iron oxide, silica, 4-N,N-(2-ethylhexyl)methylaminobenzoic acid ester of 2,4-dihydroxybenzophenone, 4-N,N-(2-ethylhexyl)-methylaminobenzoic acid ester with 4-hydroxydibenzoylmethane, 4-N,N-(2-ethylhexyl)-methylaminobenzoic acid ester of 2-hydroxy-4-(2-hydroxyethoxy)benzophenone and 4-N,N(2-ethylhexyl)-methylaminobenzoic acid ester of 4-(2-hydroxyethoxy)dibenzoylmethane. Suitable anti-acne agents include salicylic acid; 5-octanoyl salicylic acid; resorcinol; retinoids such as retinoic acid and its derivatives; sulfur-containing D and L amino acids other than cysteine; lipoic acid; antibiotics and antimicrobials such as benzoyl peroxide, octopirox, tetracycline, 2,4,4′-trichloro-2′-hydroxydiphenyl ether, 3,4,4′-trichlorobanilide, azelaic acid, phenoxyethanol, phenoxypropanol, phenoxisopropanol, ethyl acetate, clindamycin and melclocycline; flavonoids; and bile salts such as scymnol sulfate, deoxycholate and cholate. Examples of anti-wrinkle and anti-skin atrophy agents are retinoic acid and its derivatives, retinol, retinyl esters, salicylic acid and its derivatives, sulfur-containing D and L amino acids except cysteine, alpha-hydroxy acids (e.g., glycolic acid and lactic acid), phytic acid, lipoic acid and lysophosphatidic acid.

The formulations may also contain irritation-mitigating additives to minimize or eliminate the possibility of skin irritation or skin damage resulting from the permeation-enhancing base or other components of the composition. Suitable irritation-mitigating additives include, for example: alpha-tocopherol; monoamine oxidase inhibitors, particularly phenyl alcohols such as 2-phenyl-1-ethanol; glycerin; salicylic acids and salicylates; ascorbic acids and ascorbates; ionophores such as monensin; amphiphilic amines; ammonium chloride; N-acetylcysteine; cis-urocanic acid; capsaicin; and chloroquine. The irritant-mitigating additive, if present, may be incorporated into the present formulations at a concentration effective to mitigate irritation or skin damage, typically representing not more than about 20 wt. %, more typically not more than about 5 wt. %, of the composition.

A dry-feel modifier is an agent which when added to an emulsion, imparts a “dry feel” to the skin when the emulsion dries. Dry feel modifiers can include talc, kaolin, chalk, zinc oxide, silicone fluids, inorganic salts such as barium sulfate, surface treated silica, precipitated silica, fumed silica such as an Aerosil available from Degussa Inc. of New York, N.Y. U.S.A. Another dry feel modifier is an epichlorohydrin cross-linked glyceryl starch of the type that is disclosed in U.S. Pat. No. 6,488,916.

Other agents may also be added, such as antimicrobial agents, to prevent spoilage upon storage, i.e., to inhibit growth of microbes such as yeasts and molds. Suitable antimicrobial agents are typically selected from the group consisting of the methyl and propyl esters of p-hydroxybenzoic acid (i.e., methyl and propyl paraben), sodium benzoate, sorbic acid, imidurea, purite, peroxides, perborates and combinations thereof.

The compositions of the invention can include additional medicinal agents or their pharmaceutically acceptable salts. One of skill in the art can readily choose a medical agent to incorporate into the compositions of the invention and its appropriate concentration depending on the indication and desired effect. Examples of medicinal agents include, but not limited to, α2 adrenergic receptor agonist, and optionally one or more other pharmaceutically active ingredients, including, but not limited to, coal tar, dithranol (anthralin), corticosteroids like desoximetasone (Topicort), fluocinonide, vitamin D analogues (for example, calcipotriol), retinoids, Argan oil, psoralen, methotrexate, cyclosporine, retinoids or other synthetic forms of vitamin A, Amevive®, Enbrel®, Humira®, and Remicade®.

The topically administrable composition according to embodiments of the invention can further include anesthetics and analgesics, such as camphor, menthol, and pramoxine, baclofen, amitriptyline, doxepin, α2 adrenergic agents (e.g., clonidine), ketamine, glyceryl trinitrate, pimecrolimus, phenytoin, benzyl alcohol, diclofenac, salicylates, dyclonine, ethyl chloride, hexylresorcinol, and trolamine, topical opioids, capsaicin, and gabapentin or pregabalin, anesthetics such as esters, for example Benzocaine, Chloroprocaine, Cocaine, Cyclomethycaine, Dimethocaine, Larocaine, Propoxycaine, Procaine, Novocaine, Proparacaine, Tetracaine, and Amethocaine; amides, for example, Articaine, Bupivacaine, Carticaine, Cinchocaine, Dibucaine, Etidocaine, Levobupivacaine, Lidocaine, Lignocaine, Mepivacaine, Piperocaine, Prilocalne, Ropivacaine, Trimecaine; and naturally occurring anesthetics including Saxitoxin, and Tetrodotoxin, xylocaine, dyclonin, butamen picrate, dimethisoquin hydrochloride, cyclomethylcaine sulfate, and the like, and anti-inflammatory agents such as NSAIDs (e.g., diclofenac, ketoprofen, flurbiprofen, ibuprofen, naproxen, indomethacin, piroxicam) buprenorphin, butophanol tartrate, acetaminophen, fentanyl, mefenamic acid, flutenamic acid, oxyphenbutazone, phenybutazone, menthol, methyl salicylate, phenol, salicylic acid, benzyl alcohol, camphorated metacresol, juniper tar, resorcinol, allyl isothiocyanate, capsaicin, and the like; corticosteroids such as alclometasone dipropionate, amcinocide, hydrocortisone, betamethasone dipropionate, betamethasone valerate, desoximetasone, clobetasol propionate, flurandrenolide, halcinonide, halobetasol, estradiol, testosterone, progesterone, fluticasone, clobetasol, dexamethasone, dexonide, fluocinolone acetonide, flucinonide, medroxyprogesterone, mometasone furoate, triamcinolone, and the like; hormones (such as estrogens, estradiol, progesterol, progesterone, testosterone, insulin, calcitonin, parathyroid hormone, peptide and vasopressin hypothalamus releasing factor), cannabinoids such as tetrahydrocannabinol (THC) and cannabidiol (CBD), or other analgesics; antifungals, such as ciclopirox, chloroxylenol, triacetin, sulconazole, nystatin, undecylenic acid, tolnaftate, miconizole, clotrimazole, oxiconazole, griseofulvin, econazole, ketoconozole, and amphotericin B; antibiotics and anti-infectives, such as mupirocin, erythromycin, clindamycin, gentamicin, polymyxin, bacitracin, and silver sulfadiazine; and antiseptics, such as iodine, povidine-iodine, benzalkonium chloride, benzoic acid, chlorhexidine, nitrofurazine, benzoyl peroxide, hydrogen peroxide, hexachlorophene, phenol, resorcinol, and cetylpyridinium chloride.

Other compounds include additives such as muscle relaxants or cramping relief compounds such as for example, baclofen, magnesium ions (e.g., from magnesium oxide or other magnesium preparations), vitamin B1, B2, B6 and/or vitamin B12 (separately or in a complex), or compounds which aid in a person's sleep such as anti-histaminergic/inflammatory drugs (e.g., H1 receptor antagonist, diclofenac), GABA-A receptors agonists and antagonists (e.g., benzodiazepines (BZ), zolpidem (AMBIEN®), zaleplon (SONATA®), and eszopiclone (LUNESTA®), agonists for the melatonin receptors (e.g., melatonin), antidepressants (e.g., amitriptyline and doxepin), and angiotensin II type 1 (ATI) receptor antagonists. Those of skill in the art will readily recognize additional ingredients that can be admixed in the compositions described herein.

The formulation may also contain an aesthetic agent. Examples of aesthetic agents include fragrances, pigments, colorants, essential oils, skin sensates and astringents. Suitable aesthetic agents include clove oil, menthol, camphor, eucalyptus oil, eugenol, methyl lactate, bisabolol, witch hazel distillate and green tea extract.

Fragrances are aromatic substances which can impart an aesthetically pleasing aroma. Typical fragrances include aromatic materials extracted from botanical sources (i.e., rose petals, gardenia blossoms, jasmine flowers, etc.) which can be used alone or in any combination to create essential oils. Alternatively, alcoholic extracts may be prepared for compounding fragrances. However, due to the relatively high costs of obtaining fragrances from natural substances, the modern trend is to use synthetically prepared fragrances, particularly in high-volume products. One or more fragrances can optionally be included in the sunscreen composition in an amount ranging from about 0.001 to about 5 weight percent, p or about 0.01 to about 0.5 percent by weight. Additional preservatives may also be used if desired and include well-known preservative compositions such as benzyl alcohol, phenyl ethyl alcohol and benzoic acid, diazolydinyl, urea, chlorphenesin, iodopropynyl and butyl carbamate, among others.

The following examples are given in order to provide a person skilled in the art with a sufficiently clear and complete explanation of the present invention, but should not be considered as limiting of the essential aspects of its subject, as set out in the preceding portions of this description.

### EXAMPLES

### Example 1: Preparation of Pirenzepine Dihydrochloride Formulation

For a typical pirenzepine dihydrochloride composition of the present application, the pirenzepine dihydrochloride was first weighed into a suitable container and the appropriate amount of water was transferred quantitatively into the container. Citric acid, sodium citrate and sodium hydroxide were weighed and added to this water phase. The resulting mixture was stirred until dissolution was complete. Additional solvents as discussed in more detail below, such as dimethyl sulfoxide (“DMSO”), ethanol, or propylene glycol, etc. were then added and the mixture was briefly mixed. Surfactants, fatty acids, preservative and any other constituents outside of thickening agents were then added. The resulting mixture was then stirred until dissolution of all ingredients was complete. Finally, any thickening agent (such as cellulose thickeners, polyvinyl alcohol, Carbopols, etc.) were then added. The final mixture was stirred overnight to allow the thickening agent to fully swell.

### Example 2: Representative Formulation of Pirenzepine Dihydrochloride

A representative formulation (WinF54) of pirenzepine dihydrochloride was prepared (refer to Table 6 for specific amounts of the components):

1. Weigh pirenzepine dihydrochloride in a suitable container.

2. Quantitatively transfer water into the container.

3. Quantitatively add citric acid, sodium citrate anhydrous, and sodium hydroxide. Mix until complete dissolution.

4. Quantitatively add dimethyl sulfoxide, benzyl alcohol, and glycerin. Briefly mix.

5. Quantitatively add Brij S20 (Steareth-20), lauryl lactate and disodium edetate. Mix until complete dissolution.

### Example 3: Typical Procedure for Preparation of Formulation of Pirenzepine Free Base

For a typical pirenzepine free base composition of the present application, the pirenzepine free base was first weighed into a suitable container. Solvents were then added (such as dimethyl sulfoxide, ethanol, propylene glycol, water, etc.) and the mixture was mixed until dissolution was complete. Finally, surfactants, fatty acids, preservative and any other constituents were added. The final mixture was then stirred until dissolution of all ingredients was complete.

### Example 4: Representative Formulation of Pirenzepine Free Base

A representative formulation (WinFB20) of pirenzepine free base was prepared as follows. See Table 7 below for amounts of each component.

1. Weigh pirenzepine free base in a suitable container. 2. Quantitatively transfer dimethyl sulfoxide, propylene glycol, ethanol and water into the container. Using a suitable mixer, mix until complete dissolution.

3. Quantitatively add Brij S20 (Steareth-20), Brij 30 (Laureth-4) and lauryl lactate into the container.

4. Quantitatively mix until complete dissolution.

### Example 5: Other Formulations of Pirenzepine Dihydrochloride and Pirenzepine Free Base

Other formulations of both pirenzepine dihydrochloride and pirenzepine free base were made following the above mixing protocols. These formulations are outlined in Table 7.

### Example 6: Skin Permeation Measurements

The permeation of pirenzepine dihydrochloride and pirenzepine from the varying formulations was measured using Franz diffusion cells (“FDC”s). Both porcine skin and cadaver skin samples were used as the substrate for the numerous flux studies.

For the studies using porcine skin as the substrate, porcine skin pieces were obtained from Lampire Biological Laboratories, Inc. (Pipersville, Pa.). Porcine skins were collected immediately following animal sacrifice, and the hairs were trimmed with clippers. The skin was then trimmed to a set thickness of some 2 mm and stored at −20° C. until the day of the study. Prior to use, the porcine skin pieces were allowed to thaw in air, to room temperature, then dermatomed to a thickness of 1 mm and cut into circular pieces of an appropriate size prior to mounting on the FDC.

For the studies using cadaver skin as the substrate, cadaver skin pieces were obtained from New York Firefighters Tissue Bank (New York, N.Y.). Cadaver skin pieces were collected from the posterior torso from Caucasian male or females, age 30-70 years old. Cadaver skin pieces were collected and dermatomed by the tissue bank to a thickness of ˜400m. The skin pieces were set frozen and store at −20° C. until the day of the study. Prior to use, the cadaver skin pieces were allowed to thaw in air, to room temperature and cut into circular pieces of an appropriate size prior to mounting on the FDC.

The FDCs had a surface area of 0.55 cm² and a 3.3 ml receptor well volume that was filled with isotonic phosphate buffered saline (“PBS”) doped with 0.01% sodium azide and 2 wt % hydroxypropyl betacyclodextrin (“HPBCD”). The skin pieces were then mounted on the receptor well with the stratum corneum side facing up. The donor well was then aligned on top of the receptor well and the flanges of the wells were clamped together with uniform pressure using a pinch clamp (SS #18 VWR 80073-350 from VWR Scientific, West Chester Pa.). After the FDCs were assembled, the skin pieces were allowed to hydrate for 20 minutes. When cadaver skin was used as the substrate, a tritiated water pretesting of the skin was performed which consisted of measuring the flux of tritiated water across the skin over the period of an hour, and assigning FDCs to each formulation to be tested that had roughly equivalent average water flux values. FDCs with anomalously high water flux values were dropped from the study.

After the cells were assembled and tritiated water flux values were complete, the cells were dosed with 5 ml of the formulation and the formulation was spread across the skin surface. The receptor wells were maintained at 32° C. throughout the study in a stirring dry block heater with continual agitation via a stir bar. Throughout the study, 300 μl samples aliquots were collected from the receptor chamber using a syringe, and fresh buffer was replaced into the receptor chamber. Upon completion of the study, the surface of the skin was wiped with Kimwipes soaked with 50 vol % water/50 vol % ethanol to remove the residual formulation. The skin was then tapped dry and tape stripped three times (which consists of applying strips of cellophane tape to the skin and peeling them off to successively remove layers of the stratum corneum). The tape strips were discarded and the remaining skin piece was heat separated into epidermal and dermal pieces. The skin pieces were then collected into a glass vial and 2 ml of an extraction solvent (80 vol % water/20 vol % ethanol) was added to the vial. The skin pieces were then incubated for 24 hours at 40° C. with constant agitation to allow extraction of the active from the skin. After 24 hours, samples were then collected, and filtered.

Both receptor and skin samples were analyzed for pirenzepine concentration either via HPLC analysis using a C18 column with acetonitrile and water with 0.1% 10mMol NH₄HCO₃ as the mobile phase or with liquid scintillation counting (depending on whether or not C¹⁴ labelled pirenzepine dihydrochloride was used in the formulation). After all the data was collected, the flux rates were calculated and averaged across the multiple replicates tested for each formulation.

### Example 7: Comparing the Effect of Solvents Added to a Water Based Vehicle

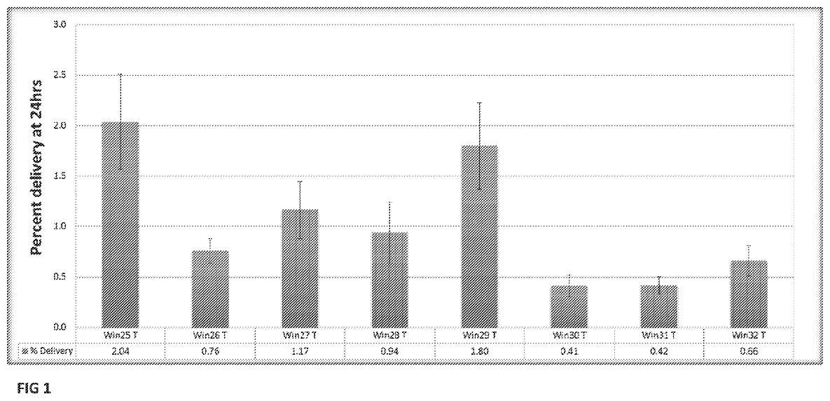

Several pirenzepine formulations (Win25T-Win32T) were prepared to compare the effects of varying solvents on the flux of pirenzepine across the skin. The formulations included 72 wt % water, 8 wt % pirenzepine dihydrochloride and 20 wt % of a varying solvents as shown below. The formulation ingredients are shown in Table 1 below. All the solvents tested were fully miscible with water. The results of this flux study are shown in FIG. 1. As can be seen from FIG. 1, dimethyl sulfoxide (Win25T) and diethylene glycol monoethyl ether (Win29T) (TRANSCUTOL®) had the greatest effect in increasing the flux of pirenzepine across the skin. Win25T resulted in 2.04% of the pirenzepine delivered across the skin, while Win29T resulted in 1.8% of the pirenzepine delivered across the skin.

TABLE 1
Pirenzepine dihydrochloride formulations of various solvents added to a water based vehicle.

| | Formulation name | | | | | | | |
|---|---|---|---|---|---|---|---|---|
| | Win25T | Win26 T | Win27T | Win28T | Win29T | Win30T | Win31T | Win32T |
| Ingredient | mg | mg | mg | mg | mg | mg | mg | mg |
| Water | 72 | 72 | 72 | 72 | 72 | 72 | 72 | 72 |
| Pirenzepine | 8 | 8 | 8 | 8 | 8 | 8 | 8 | 8 |
| dihydrochloride | | | | | | | | |
| Propylene glycol | | 20 | | | | | | |
| Polythylene glycol 400 | | | | | | 20 | | |
| Transcutol ® | | | | | 20 | | | |
| (diethylene glycol | | | | | | | | |
| monoethyl ether) | | | | | | | | |
| Hexylene glycol | | | | | | | 20 | |
| Dimethyl sulfoxide | 20 | | | | | | | |
| Ethanol | | | 20 | | | | | |
| Isopropyl alcohol | | | | | | | | 20 |
| Dimethyl isosorbide | | | | 20 | | | | |

### Example 8: Comparing Surfactants in a Water Plus DMSO Vehicle

Several pirenzepine formulations (Win36T— Win49T) were prepared to compare the effects of varying surfactants and solvents on the flux of pirenzepine across the skin. The formulations were compared to a control formulation consisting of 90 wt % water and 10 wt % pirenzepine dihydrochloride. The test formulations had a base water/DMSO/pirenzepine dihydrochloride vehicle and added in additional surfactants and solvents.

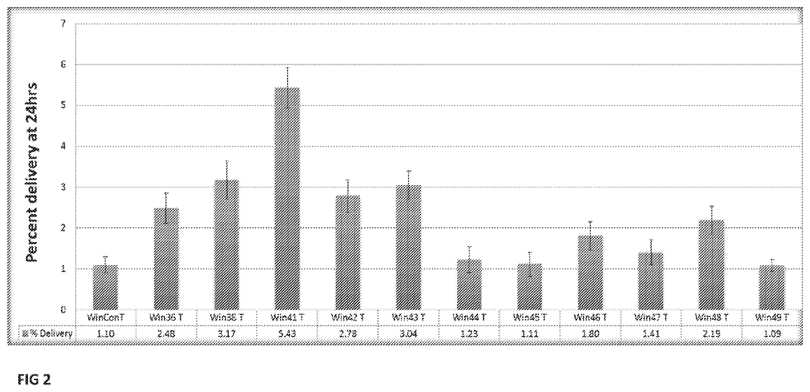

The formulation ingredients used in this example are shown below in Table 2. The results of this flux study are shown in FIG. 2. Several conclusions can be drawn by looking at FIG. 2: (1) the addition of Brij™ 30 increased the transdermal flux of pirenzepine (comparing Win38T to Win36T), (2) this effect is most noticeable in combination with 20% DMSO as opposed to 10 wt % DMSO with 10 wt % of another solvent (comparing Win41T to Win38T, Win42T and Win43T, (3) the addition of other surfactants did not increase the flux of pirenzepine across the skin (comparing Win44T to Win36T). Brij™ 30 is a Polyoxyethylene(4)lauryl ether.

TABLE 2
Formulations investigating the effect of various surfactants added to a water and DMSO based vehicle.

| | mg | mg | mg | mg | mg | mg | mg | mg | mg | mg | mg | mg |
|---|---|---|---|---|---|---|---|---|---|---|---|---|
| | Formulation name | | | | | | | | | | | |
| Ingredient | WinConT | Win36T | Win38T | Win41T | Win42T | Win43T | Win44T | Win45T | Win46T | Win47T | Win48T | Win49T |
| Sodium laureth sulfate | | | | | | | | | | | | 7.5 |
| (70% by weight) | | | | | | | | | | | | |
| Sodium docusate | | | | | | | | | | | 5 | |
| Phenoxyethanol | | | | | | | | | | 2.5 | | |
| Span20 | | | | | | | | | 5 | | | |
| N-lauryl sarcosinate | | | | | | | | 5 | | | | |
| Sodium sulfoacetate | | | | | | | 5 | | | | | |
| Transcutol | | | | | | 10 | | | | | | |
| Ethanol | | | | | 10 | | | | | | | |
| Brij30 | | | 5 | 5 | 5 | 5 | | | | | | |
| Dimethyl isosorbide | | 10 | 10 | | | | 10 | 10 | 10 | 10 | 10 | 10 |
| Dimethyl sulfoxide | | 10 | 10 | 20 | 10 | 10 | 10 | 10 | 10 | 10 | 10 | 10 |
| Transcutal | | | | | | | | | | | | |
| Pirenzepine dihydrochloride | 10 | 8 | 8 | 8 | 8 | 8 | 8 | 8 | 8 | 8 | 8 | 8 |
| Water | 90 | 72 | 72 | 72 | 72 | 72 | 72 | 72 | 72 | 72 | 72 | 72 |

### Example 9: Screening Enhancers in Combination with DMSO, Dimethyl Isosorbide and Brij

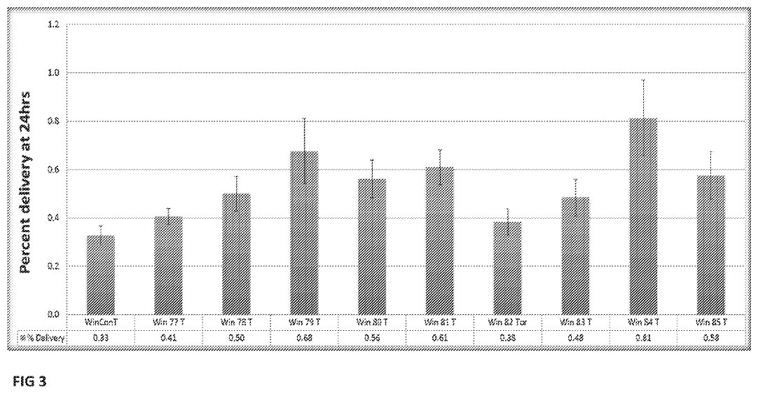

Several pirenzepine formulations (Win77T-Win85T) were prepared to compare the effects of varying penetration enhancers on the flux of pirenzepine across the skin. The formulations were compared to a control formulation consisting of 90 wt % water and 10 wt % pirenzepine dihydrochloride. The test formulations had a base water/DMSO/Dimethyl isosorbide/Brij 78/pirenzepine dihydrochloride vehicle and added in additional penetration enhancers. The formulation ingredients are shown in Table 3. The results of this flux study are shown in FIG. 3. Several conclusions can be drawn by looking at FIG. 3: (1) the addition of Brij™ 78 increases the transdermal flux of pirenzepine (comparing Win78T to Win77T) confirming previous Brij effects, (2) the addition of lauryl lactate, benzyl alcohol or glyceryl monooleate further increases the flux of pirenzepine, (3) the addition of other surfactants did not increase the flux of pirenzepine across the skin (comparing Win44T to Win36T).

TABLE 3
Formulations investigating the effect of various penetration enhancers
added to a water/DMSO/dimethyl isosorbide/Brij78 base.

| | Formulation name | | | | | | | | | |
|---|---|---|---|---|---|---|---|---|---|---|
| | WinConT | Win77T | Win78T | Win79T | Win80T | Win81T | Win82T | Win83T | Win84T | Win85T |
| Ingredient | mg | mg | mg | mg | mg | mg | mg | mg | mg | mg |
| Water | 90 | 72 | 72 | 72 | 72 | 72 | 72 | 72 | 72 | 72 |
| Pirenzepine dihydrochloride | 10 | 8 | 8 | 8 | 8 | 8 | 8 | 8 | 8 | 8 |
| Dimethyl sulfoxide | | 10 | 10 | 10 | 10 | 10 | 10 | 10 | 10 | 10 |
| Dimethyl isosorbide | | 10 | 10 | 10 | 10 | 10 | 10 | 10 | 10 | 10 |
| Brij78 | | | 5 | 5 | 5 | 5 | 5 | 5 | 5 | 5 |
| Benzyl alcohol | | | | 3 | | | | | | |
| Lauryl lactate | | | | | 3 | | | | | |
| Isopropyl myristate | | | | | | 3 | | | | |
| Methyl salicylate | | | | | | | 3 | | | |
| Capric triglycerides | | | | | | | | 3 | | |
| Glyceryl oleate | | | | | | | | | 3 | |
| Oleyl alcohol | | | | | | | | | | 3 |

### Example 10: Screening Enhancers in Combination with DMSO

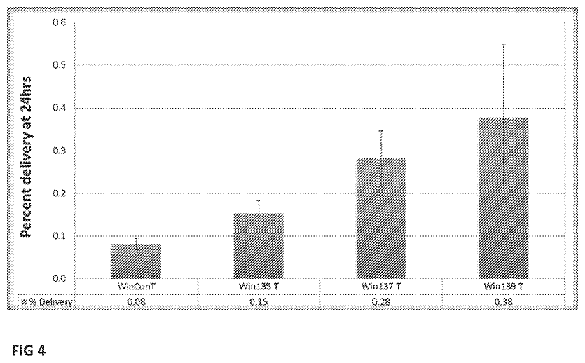

Several pirenzepine formulations (Win135T, 137T, and 139T) were prepared to further compare the effects of varying excipients on the flux of pirenzepine across the skin. The formulations were compared to a control formulation consisting of 90 wt % water and 10 wt % pirenzepine dihydrochloride. The formulation ingredients are shown in Table 4. The results of this flux study are shown in FIG. 4. The results of this study confirm previously observed results: (1) the addition of DMSO increases flux over a base water vehicle (comparing Win135T to WinConT), (2) the addition of a Brij surfactant in combination with DMSO increases flux (compare Win137T to Win135T), (3) the addition of lauryl lactate further increases flux (compare Win139T to Win137T).

TABLE 4
Formulations investigating the effect of various
penetration enhancers added to a water base.

| | Formulation name | | | |
|---|---|---|---|---|
| | WinConT | Win135T | Win137T | Win139T |
| Ingredient | mg | mg | mg | mg |
| Water | 90 | 72 | 72 | 72 |
| Pirenzepine | 10 | 8 | 8 | 8 |
| dihydrochloride | | | | |
| Dimethyl sulfoxide | | 20 | 20 | 20 |
| Brij78 | | | 5 | 5 |
| Benzyl alcohol | | 3 | 3 | 3 |
| Lauryl lactate | | | | 3 |

### Example 11: Screening Enhancers in Gel Formulations

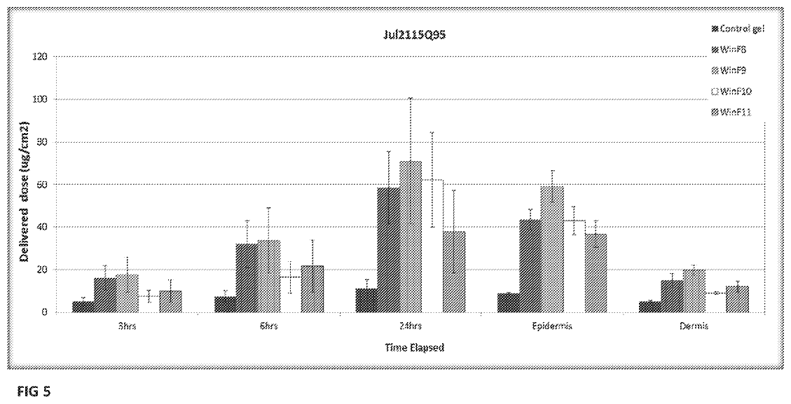

Pirenzepine formulations (WinF8-WinF11) were prepared to verify that the penetration enhancement effect of the previously identified excipients worked in a gel format. The gel formulations were compared to a control pirenzepine dihydrochloride gel. The formulation ingredients are shown in Table 5. The results of this flux study are shown in FIG. 5. The results of this study confirmed previously observed results in that the addition of DMSO with Brij and lauryl lactate increases the flux of pirenzepine across the skin. The addition of capric triglycerides in place of lauryl lactate also worked equal well in increasing the flux rate.

TABLE 5
Formulations investigating the effect of various
penetration enhancers added to a water base.

| | Formulation name | | | | |
|---|---|---|---|---|---|
| | Control | | | | |
| | Gel | WinF8 | WinF9 | WinF10 | WinF11 |
| Ingredient | mg | mg | wt/wt % | wt/wt % | wt/wt % |
| Pirenzepine | 13.83 | 11.06 | 11.06 | 11.06 | 11.06 |
| dihydrochloride | | | | | |
| Water | 76.45 | 61.16 | 61.16 | 61.16 | 61.16 |
| Glycerin | 4.41 | 3.53 | 3.53 | 3.53 | 3.53 |
| Sodium hydroxide | 1.32 | 1.06 | 1.06 | 1.06 | 1.06 |
| Citric acid, anhydrous | 0.41 | 0.33 | 0.33 | 0.33 | 0.33 |
| Sodium citrate, | 1.97 | 1.58 | 1.58 | 1.58 | 1.58 |
| anhydrous | | | | | |
| Methocel E4M (HPMC) | 1.5 | 1.20 | 1.20 | 1.20 | 1.20 |
| Benzalkoniumchloride | 0.01 | 0.01 | 0.01 | 0.01 | 0.01 |
| Disodium edetate | 0.1 | 0.08 | 0.08 | 0.08 | 0.08 |
| DMSO | | 20.0 | 20.0 | 20.0 | 20.0 |
| Brij30 | | 5.0 | 5.0 | 5.0 | 5.0 |
| Benzyl Alcohol | | 3.0 | 3.0 | 3.0 | 3.0 |
| Lauryl Lactate | | 3.0 | | | |
| Capric Triglycerides | | | 3.0 | | |
| Oleyl Alcohol | | | | 3.0 | |
| Cedar Leaf Oil | | | | | 3.0 |

### Example 12: Additional Screening of Enhancers in Gel Formulations

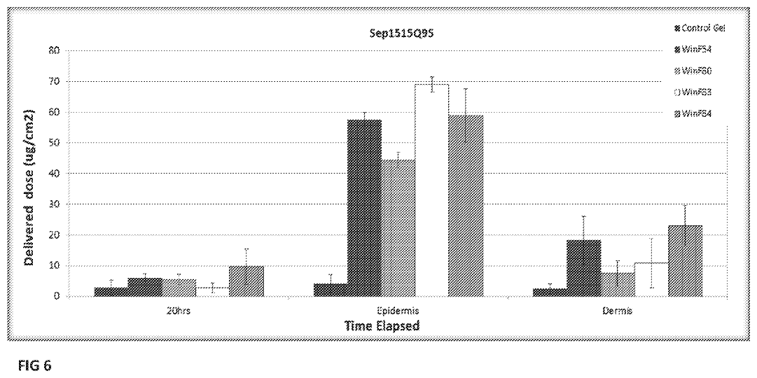

Pirenzepine formulations (WinF54, WinF80, WinF83, and WinF84) were prepared to verify the penetration enhancement effect of the previously identified excipients in gel formulations when compared to a control pirenzepine dihydrochloride gel. The formulation ingredients are shown in Table 6. The results of this flux study are shown in FIG. 6.

TABLE 6
Gel formulations (with the exception of WinF54 which
is a liquid) investigating the effect of various
penetration enhancers added to a water base.

| | Formulation name | | | | |
|---|---|---|---|---|---|
| | Control | | | | |
| | Gel | WinF54 | WinF80 | WinF83 | WinF84 |
| Ingredient | mg | mg | mg | mg | mg |
| Pirenzepine | 13.83 | 9.25 | 11.06 | 11.06 | 11.06 |
| Dihydrochloride | | | | | |
| Water | 76.45 | 52.175 | 61.16 | 61.16 | 61.16 |
| Glycerine | 4.41 | 3.0 | 3.53 | 3.53 | 3.53 |
| NaOH | 1.32 | 0.9 | 1.06 | 1.06 | 1.06 |
| Ctric acid, an | 0.41 | 0.275 | 0.33 | 0.33 | 0.33 |
| Sodium citrate, | 1.97 | 1.3 | 1.58 | 1.58 | 1.58 |
| anhydrous | | | | | |
| Disodium edetate | 0.1 | 0.1 | 0.08 | 0.08 | 0.08 |
| Benzalkonium | 0.01 | | | | |
| chloride | | | | | |
| Methocel E4M (HPMC) | 1.5 | | 1.0 | 1.0 | 1.0 |
| DMSO | | 24.5 | 5.0 | 5.0 | 15.0 |
| Benzyl Alcohol | | 3.0 | 3.0 | 3.0 | 3.0 |
| Brij30 | | 4.0 | 4.0 | 4.0 | 4.0 |
| BrijS20 | | | 4.0 | 4.0 | 4.0 |
| Lauryl lactate | | 1.5 | 2.0 | 2.0 | |
| Propylene Glycol | | | | 10.0 | 10.0 |
| Transcutol | | | 20.0 | | |
| Ethanol | | | | 25.0 | 25.0 |

### Example 13: Screening Pirenzepine Free Base Formulations Versus Pirenzepine Dihydrochloride Formulations

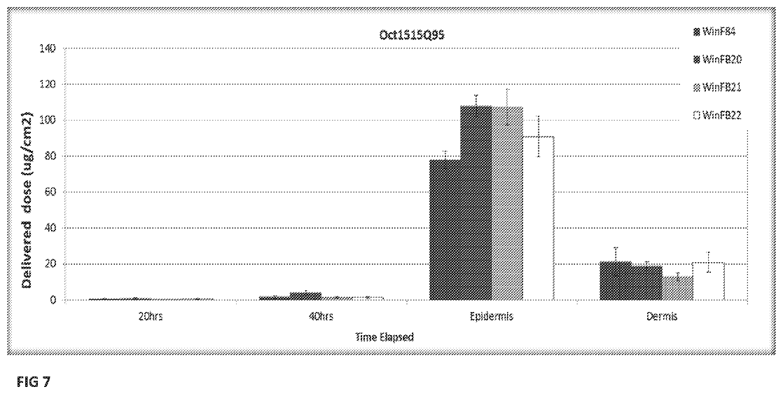

Pirenzepine free base formulations (WinFB20, WinFB21, and WinFB22) were prepared and compared to a previously prepared pirenzepine dihydrochloride gel (WinF84). The formulation ingredients are shown in Table 7. The results of this flux study are shown in FIG. 7. The results of this study show that the excipients previously shown to increase the flux of pirenzepine dihydrochloride, also work in conjunction with pirenzepine free base.

TABLE 7
Pirenzepine free base formulations

| | Formulation name | | | |
|---|---|---|---|---|
| | WinF84 | WinFB20 | WinFB21 | WinFB22 |
| Ingredient | mg | mg | mg | mg |
| Pirenzepine dihydrochloride | 11.06 | | | |
| Pirenzepine free base | | 5.0 | 5.0 | 5.0 |
| Water | 61.16 | 10.0 | 10.0 | 10.0 |
| Glycerine | 3.53 | | | |
| NaOH | 1.06 | | | |
| Citric acid, anhydrous | 0.33 | | | |
| Sodium citrate, anhydrous | 1.58 | | | |
| Disodium edetate | 0.08 | | | |
| Dimethyl sulfoxide | 15.0 | 20.0 | 20.0 | 10.0 |
| Benzyl alcohol | 3.0 | | | |
| Brij30 | 4.0 | 4.0 | 4.0 | |
| BrijS20 | 4.0 | 4.0 | 4.0 | |
| Lauryl Lactate | | 2.0 | 2.0 | |
| Propylene glycol | 10.0 | 40.0 | 40.0 | 40.0 |
| Oleyl alcohol | | | | 5.0 |
| Levulinic acid | | | | 3.0 |
| Ethanol | 25.0 | 40.0 | 40.0 | 40.0 |
| Dimethyl isosorbide | | | | 10.0 |
| Methocel E4M (HPMC) | 1.0 | | 1.0 | |

### Example 14: Pharmacokinetic Report: A Randomized, Double-Blind, Placebo-Controlled, Multiple Dose, Safety, Tolerability and Pharmacokinetic Study of Topical Pirenzepine in Healthy Volunteers

### 14.1—Example 14 Abbreviations

Table 8 includes abbreviations used in Example 14.

TABLE 8
Abbreviations

| | |
|---|---|
| AUC_{0-tau Dx} | Area under the plasma concentration-time curve from time |
| | 0 to the end of the dosing interval, on Day x where |
| | “x” is 1, 7 or 14 |
| AUC_{INF, D14} | Area under the plasma concentration-time curve from time |
| | 0 to infinity, Day 14 |
| AUC_{0-tau, D14} | Area under the plasma concentration-time curve following |
| | final dose to the end of the dosing interval) |
| BLOQ | Below the limit of quantitation |
| C_{avg, D7} | Average plasma concentration over the dosing interval on |
| | Day x where x is 7 or 14 |
| C_{max, Dx} | Maximum observed plasma concentration, on Day x where |
| | “x” is 1, 7 or 14 |
| C_{min, Dx} | Minimum plasma concentration following dose on Day x |
| | where “x” is 7 or 14 |
| Cₜₐᵤ | Plasma concentration at the end of the dosing interval |
| C_{trough, Dx} | Plasma concentration observed just before treatment |
| | administration during repeated dosing for |
| | Day 7 and Day 14 |
| Fluct_{, D7} | Percent fluctuation for steady state data |
| IV | Intravenous |
| kel | The first-order terminal elimination rate constant |
| LLOQ | Lower limit of quantitation |
| PD | Pharmacodynamic |
| PK | Pharmacokinetic |
| SAP | Statistical Analysis Plan |
| SD | Standard deviation |
| t_{1/2} | The apparent terminal elimination half-life, |
| t_{max, Dx} | The time to maximum plasma concentration on Day x |
| | where “x” is 1, 7 or 14 |
| ULOQ | Upper limit of quantitation |

### 14.2—Example 14 Objectives

To evaluate and compare the pharmacokinetic (PK) profile of three different pirenzepine formulations (WinFB34, WinF90 and WinFB100) after daily topical dose administration in healthy subjects for 14 days.

An exploratory objective of this study was to characterize the drug concentration data acquired from skin biopsies (from Cohorts 2 and 3) collected on Day 14 and to investigate potential relationships relating to relative absorption (biopsy from treated skin) and relative distribution (biopsy from untreated skin).

### 14.3—Example 14 Study Design

### 14.3.1—Overview

In this single center, double-blind, randomized, placebo-controlled, multiple dose study; consisting of a total of 24 healthy subjects: 3 cohorts of 8 subjects (6 subjects assigned to pirenzepine: 2 assigned to placebo in each cohort); each cohort was administered different topical formulations of pirenzepine: WinFB34, WinF90 and WinFB100.

The following pirenzepine topical formulations or matching placebos were applied to the lower extremities (calves, ankles, and tops of feet in an area of ˜750 cm² on each leg, total dose applied to 2 legs), as follows:
Cohort 1: 4.0% pirenzepine topical solution WinFB34 or matching placebo—2.5 mL applied once daily for Days 1-7 and 5.0 mL applied once daily on Days 8-14.
Cohort 2: 6.5% pirenzepine topical solution WinF90 or matching placebo—2.5 mL applied once daily for Days 1-7 and 5.0 mL applied once daily on Days 8-14.
Cohort 3: 4.0% pirenzepine topical solution WinFB100 or matching placebo—2.5 mL applied once daily for Days 1-7 and 5.0 mL applied once daily on Days 8-14.

A dose volume of 5 mL was considered a maximum volume which could be applied in a controlled manner, but some embodiments envision the use of over 5 mL.

### 14.3.2—Sampling Times

PK: Blood samples were collected for the determination of pirenzepine concentration at the following nominal times:

Day 1 Pre-dose, 0.5, 1, 1.5, 2, 3, 4, 6, 8, 12 hours
Day 2 24 hours
Day 3 Within 30 minutes of Dose 4
Day 7 Pre-dose 7, then at 0.5, 1, 1.5, 2, 3, 4, 6, 8, 12 hours

### Day 8 Pre-dose 8

### Day 10 Pre-dose 10

Day 14 Pre-dose 14, then at 0.5, 1, 1.5, 2, 3, 4, 6, 8, 12
Day 15 24 hours post-dose 14
Day 16 48 hours post-dose 14

Blood samples on Days 1, 7, and 14 were collected via either the indwelling intravenous (IV) cannula to reduce the frequency of direct venipuncture (at the discretion of the Investigator).

Skin Biopsies: Two standard 5 mm skin biopsies were collected on Day 14 (Cohort 2 and 3 only), one from the treated area on the distal leg or calf; approximately 10 cm above the malleolus and 3 cm to the posterior; and one from the untreated area on the lateral thigh, approximately 25 cm below the iliac spine, approximately 5 cm below the level of the pubis and 8 cm to the posterior.

### 14.4—Example 14 Bioanalysis

A validated liquid chromatography tandem mass-spectroscopy method (LLOQ: 0.1 ng/mL and ULOQ: 200 ng/mL) was used to determine the concentration of pirenzepine in plasma. See TetraQ, Bioanalytical Sample Analysis Report, November, 2018. For the pirenzepine concentration analysis in biopsy samples, a qualified method (LLOQ 1.00 mg/mL) was used.

### 14.5—Example 14 Pharmacokinetic Analysis: Methods and Procedures

Pirenzepine concentrations in plasma and skin biopsies were evaluated using Phoenix™ WinNonlin® version 8.0 (Pharsight Corporation, USA) and Microsoft® Excel® 2010 (Microsoft Corporation, USA).

Pharmacokinetic Population: According to the statistical analysis plan, the PK population comprised all randomized subjects with evaluable plasma concentrations of pirenzepine (ie, subjects with plasma concentrations considered sufficient and interpretable [at the discretion of the pharmacokineticist]). Subjects who received placebo were excluded from the PK population. Any exclusion from the PK population was determined by the pharmacokineticist on blinded data, after database lock and prior to unblinding the study. A Note-to-File was prepared after unblinding to take into account all placebo samples and to exclude those placebo subjects from the PK population. The PK population was based on the actual treatment received, if that differed from that to which the subject was randomized. This population was used for the summaries and analyses of all PK data.

### 14.5.1—PK Parameters

It was apparent after Cohort 1 that there were no quantifiable drug levels for the desmethyl metabolite of pirenzepine and for that reason, PK parameters were calculated for the parent compound (outlined in Table 9 and Table 10).

TABLE 9
Primary PK Parameters

| Parameter | Definition | Method of Determination |
|---|---|---|
| C_{oax, D1,} C_{max, D7,} C_{max, D14} | maximum observed plasma | Observed from plasma concentration |
| | concentration, Day 1, Day | time profiles |
| | 7 and Day 14 | |
| C_{min, D7,} C_{min, D14} | minimum plasma concentration | |
| | following final dose on Day 7 | |
| | and Day 14 | |
| t_{max, D1,} t_{max, D7,} t_{max, D14} | the time to maximum plasma | |
| | concentration on Day 1, 7 | |
| | and 14 | |
| AUC_{0-tau, D1,} AUC_{0-tau, D7,} | area under the plasma | calculated by the linear up-log |
| AUC_{0-tau, D14} | concentration-time curve from | down trapezoidal method |
| | time 0 to the end of the | |
| | dosing interval on Day 1, 7 | |
| | or 14 | |
| AUC_{INF, D14} | Area under the plasma | calculated using the formula: |
| | concentration-time curve from | AUC_{0-inf, D14} = AUC_{0-tau, D14} + C_{last, D14}/kel, |
| | time 0 to infinity, Day 14 | D14 If the percentage of AUC_{INF} |
| | | based on extrapolation is >20%, |

TABLE 10
Secondary PK Parameters

| Parameter | Definition | Method of Determination |
|---|---|---|
| kₑₗ | first-order terminal-phase | calculated as the negative of the |
| | rate constant | slope of the terminal log-linear |
| | | segment of the plasma |
| | | concentration-time curve; |
| t_{1/2} | apparent terminal elimination | The elimination half-life (t_{1/2}) will |
| | half-life | be calculated according to the |
| | | following equation: t_{1/2} = ln (2)/kel |
| Cₜₐᵤ | plasma concentration at the | Observed from individual |
| | end of the dosing interval | concentration-time data |
| C_{trough}(D7 & D14) | plasma concentration observed | observed |
| | just before treatment | |
| | administration during repeated | |
| | dosing for Day 7 and Day 14 | |
| C_{avg, D7} | average plasma concentration | computed as AUC(0-τ) divided by |
| | over the dosing interval on | tau |
| | Day 7) | |
| C_{avg, D14} | average plasma concentration | |
| | over the dosing interval on | |
| | Day 14 | |
| Fluct_{, D7} | Percent fluctuation for steady | Fluct, _{D7} = 100 × [(C_{max, D7} − C_{min, D7})/C_{avg, D7}] |
| | state data | |
| Fluct, _{D14} | Percent fluctuation for steady | Fluct, _{D14} = 100 × [(C_{max, D14} − C_{min, D14})/C_{avg, D14}] |
| | state data | |
| Accumulation | | Day 1 and Day 14, calculated as |
| Index | | 1/(1−e\hat{ } (−(kel × tau)), where tau = 24 hours |

### 14.5.2—Analysis Procedures

Plasma:
Actual times were used for PK determinations, calculated with respect to the dose time.
Time points preceding Cₘₐₓ that were BLOQ were set to zero for PK parameter determination Where the final plasma concentration was BLOQ, the concentration at the time point preceding this value was deemed to be the last measurable concentration for calculation of kel.
In instances where % AUCexp was >20%, PK parameters which were dependent upon accurate characterisation were not calculated (i.e.) kel, t_{1/2}, AUC_{INF}.
kel was determined by log-linear regression obtained on at least the 3 last quantifiable concentrations and did not include Cₘₐₓ; the range of data used was determined by visual inspection of a semi-logarithmic plot of concentration vs. time. The goodness of fit of the regression was evaluated using the adjusted square of the correlation coefficient (R2 adjusted). The adjusted R2 of the regression line through the data points had to be at least 0.8500 for the kel value to be considered reliable.

Biopsy: The frozen 5 mm skin biopsies, were bisected in half, resulting in two sections per biopsy: the Upper Half and the Lower Half.

The two sections of the biopsy samples can be roughly divided into zones based on textbook descriptions of skin layer thicknesses (see See Noisakran S, Onlamoon N, Songprakhon P et al., Cells in Dengue Virus Infection In Vivo. Advances in Virology, 2010 (5) article ID: 164878, researchgate.net/publication/221830194_Cells_in_Dengue_Virus_Infection_In_Vivo, accessed Nov. 19, 2018):
Upper Half: containing epidermis and dermis
Lower Half: containing dermis and adipose.

Both halves of each biopsy were then homogenized and processed to determine the concentration of the pirenzepine in the treated versus untreated skin.

General assessments can be made about the gradient of distribution of pirenzepine from top to bottom resulting for a topical administration of WinF90 and WinFB100 by analyzing the upper half versus the lower half of the treated skin biopsies. A basic understanding of the distribution of pirenzepine away from the locally treated areas can be gained by comparing the concentrations of pirenzepine recovered in the treated skin versus the untreated skin biopsies.

The concentration of pirenzepine (ng/mL) in the skin biopsies was compared to the subject plasma concentration level at the collection time point (8h post dose) collected closest to the collection time point of the biopsies.

### 14.6—Example 14 Results

### 14.6.1—Plasma Pharmacokinetics

Entire Profile: The plasma PK profile for all pirenzepine formulations (WinFB34, WinF90 and WinFB100) resulted in plasma levels of less than 1.7 ng/mL pirenzepine. As the relative exposure of each parent compound was low, accordingly, the primary metabolite, desmethyl pirenzepine, was not quantifiable for any subject, at any time point, on any day, and therefore was not included in any subsequent PK analysis. By Day 14 all subjects in the active dose groups for all cohorts had quantifiable drug levels. However, the high degree of variability between subjects within each Cohort, did not allow for statistical significance to be determined for the respective PK measures. Comparison between the three pirenzepine formulations did not satisfy the criteria for relative bioavailability. In all cases, the 90% CIs for the ratio of population geometric means between each contrast was not contained within the equivalence limits of 80% to 125%. In addition, no conclusions could be made with respect to gender and demographics due to high levels of variability.

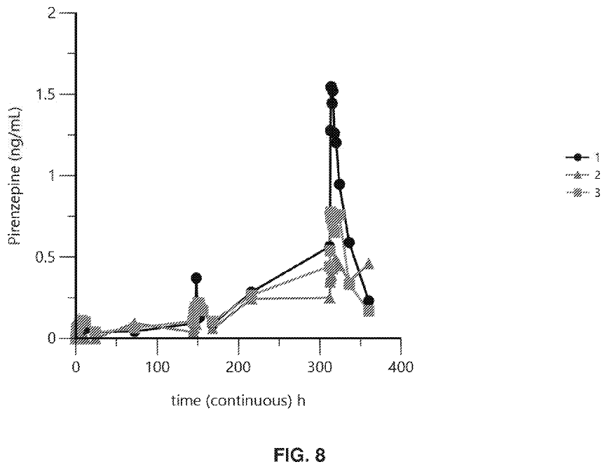

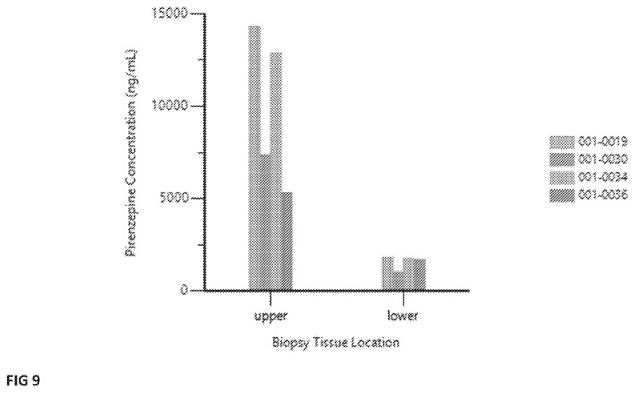

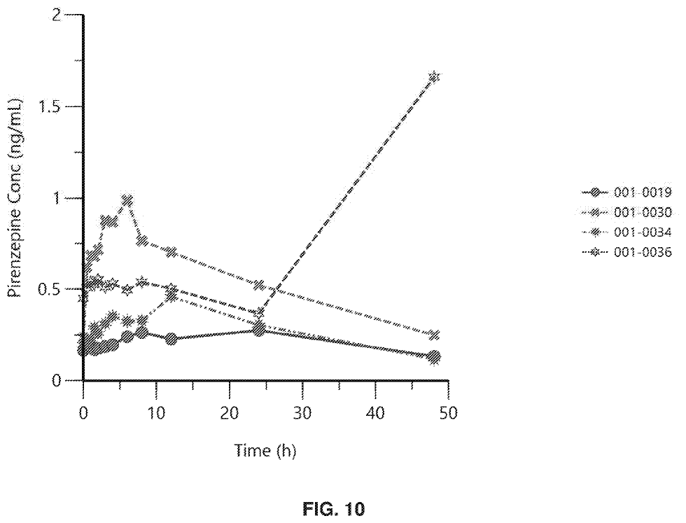

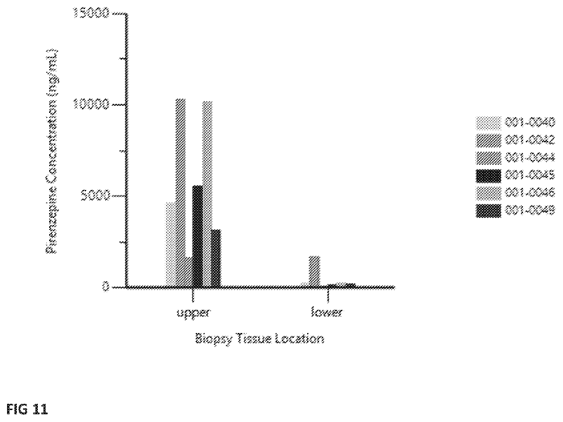

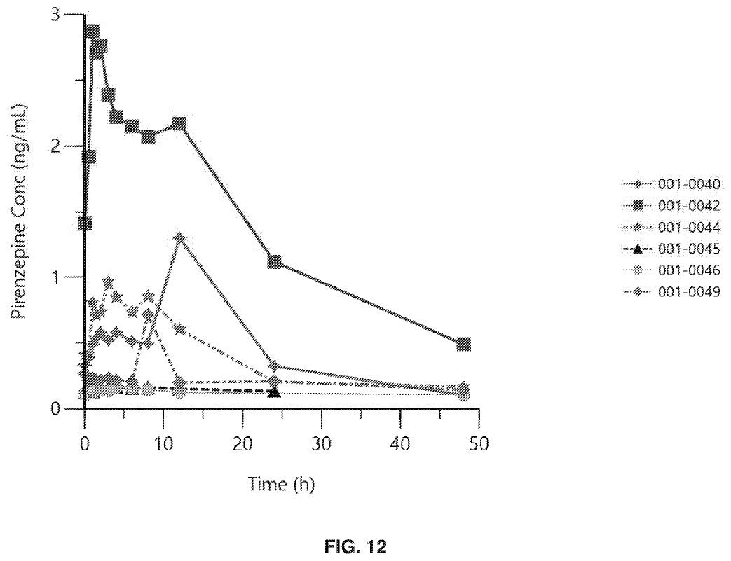

Comparison of mean profiles revealed a rank order of WinFB34>WinFB100>WinF90 in terms of C_{max, D14}, AUC_{0-tau, D14} and AUC_{inf, D14} (FIG. 8, Table 11).

T_{max, D1} occurred between 2-8 h, 4 h, 1-12 h for WinFB34, WinF90 and WinFB100, respectively while T_{max, D14} was 1.5-6 h, 2-24, 1-12 h for WinFB34, WinF90 and WinFB100, respectively. No statistical difference between any of the cohorts for T. on Days 1, 7 or 14 was observed. Similarly, no statistically significant treatment differences were observed for Cₘₐₓ or exposure parameters (AUC).

The t_{1/2} was relatively consistent across all cohorts with mean values ranging between 27 to 48 h (Table 12). C_{avg, D7} was similar for all formulations (Table 10). Following the planned increase in dose volume (5 mL daily from Day 8), both WinF90 and WinFB100, AUC_{0-tau D14} was approximately double that of Day 7, which was proportional to the doubling of the dose volume from Day 7 onwards (Table 9).

By Day 7, a tendency for a two-fold accumulation in pirenzepine plasma levels was observed (WinFB100, n=3), however this was based on limited samples for WinFB34 (n=2) and WinF90 (n=1) (Table 10, FIG. 8). Mean (SD) accumulation index was 2.211 (1.1208), 2.204 (0.4837) and 3.452 (2.3159) for Cohorts WinFB34, WinF90 and WinFB100, respectively.

By Cohort/Formulation: Cohort 1 (WinFB34): On separate consideration of the intensive PK sampling days (1, 7 and 14), given the lower starting dose (2.5 mL per day for 7 days), Days 1 and 7 plasma pirenzepine levels were lower than Day 14. On Day 1, Cohort 1 (WinFB34) (subjects 001-0002, 001-0005 and 001-0013) plasma pirenzepine levels were less than 0.25 ng/mL and by Day 7, all six subjects within the cohort had detectable pirenzepine levels, with pirenzepine levels spiking at 1.53 ng/mL at 4 h post-dose for subject 0007. By Day 14, subject 001-0005 had the highest levels (approx. 5 ng/mL) followed by subject 001-0001 (approx. 2 ng/mL). All others within Cohort 1 were less than 1.2 ng/mL.

For Cohort 2 (WinF90) on Day 1, one single point for one subject (Subject 001-0026) was detected (0.692 ng/mL at 4 h post-dose) with all other subjects within the cohort at BLOQ. By Day 7, all six subjects had detectable pirenzepine levels with the highest levels reported for subject 001-0028, (0.688 ng/mL at 1 h post-dose), subject 001-0019 (0.671 ng/mL at 3 h post-dose), subject 001-0034 (0.403 ng/mL at 2 h post-dose) with the remainder of the subjects at less than 0.3 ng/mL. By Day 14, 5 subjects had detectable levels (subject 001-0026 was BLOQ) with subject 001-0030 up to 0.987 ng/mL at 6 h post-dose and others at 0.5 ng/mL or less. Of note, subject 001-0036 spiked at 1.66 ng/mL at 48 h post dose, it is unclear if this result is an analytical artefact, a result of sample contamination or a real result.

For Cohort 3 (WinFB100), on Day 1, subjects 001-0040, 001-0044, 001-0049 had pirenzepine plasma levels less than 0.542 ng/mL. By Day 7, 5 subjects had pirenzepine levels less than 0.45 ng/mL. By Day 14, all six subjects had levels of pirenzepine; with the highest levels at 2.87 ng/mL at 1 h post-dose (Subject 001-0042).

After 14 days of topical administration mean pirenzepine levels were according to the following rank order: Cohort 1>Cohort 3>Cohort 2. An approximate accumulation factor of 2 was noted for all formulations. This was based upon the t_{1/2} which ranged from mean (SD) of 24.391 (19.0681) h, 27.453 (8.2823) h and 48.232 (39.1123) h, for Cohorts 1, 2 and 3, respectively.

TABLE 11
Summary of Primary PK parameters

| | Cohort 1 | | Cohort 2 | | Cohort 3 | |
|---|---|---|---|---|---|---|
| Parameter | WinFB34 | | WinF90 | | WinFB100 | |
| C_{max, D1} | 0.186 | (0.0376) * | NA | | 0.330 | (0.3005) |
| C_{max, D7} | 0.450 | (0.5379) | 0.427 | (0.2060) | 0.310 | (0.1086) |
| C_{max, D14} | 1.670 | (18087) | 0.556 | (0.2628) | 1.031 | (1.0051) |
| C_{min, D7} | 0.083 | (0.0662) | 0.033 | (0.0804) | 0.135 | (0.0353) |
| C_{min, D14} | 0.534 | (0.5960) | 0.233 | (0.0791) | 0.396 | (0.5024) |
| T_{max, D1} | 8 | (2, 8) | 4 | (4, 4) | 8 | (1, 12) |
| T_{max, D7} | 3.5 | (1, 12) | 4.5 | (1, 12) | 4 | (3, 8) |
| T_{max, D14} | 4 | (1.5, 6) | 8 | (2, 24) | 3.5 | (1, 12) |
| AUC_{0-tau, D1} | 2.563 | (0.6110) | NA | | 9.510** | |
| AUC_{0-tau, D7} | 5.690 | (0.4757) | 5.324 | (1.3520) | 5.611 | (1.7781) |
| AUC_{0-tau, D14} | 24.100 | (25.7022) | 10.058 | (4.3008) | 15.234 | (16.3922) |

| | | | | | |
|---|---|---|---|---|---|
| AUC_{0-∞, D14} | 51.30 | (45.452) | 16.82** | 41.78 | (31.442) |

Tₘₐₓ values Mean (Min, Max)
* n = 3,
**n = 1

TABLE 12
Summary of Secondary PK parameters

| | Cohort 1 | | Cohort 2 | | Cohort 3 | |
|---|---|---|---|---|---|---|
| Parameter | WinFB34 | | WinF90 | | WinFB100 | |
| kₑₗ | 0.03286 | (0.014074) | 0.02700 | (0.007532) | 0.02657 | (0.019566) |
| t_{1/2} | 27.391 | (19.0681) | 27.453 | (8.2823) | 48.232 | (39.1123) |
| C_{tau, D1} | 0.094 | (0.0347) * | | | 0.216** | |
| C_{tau, D7} | 0.172 | (0.0085) * | 0.178 | (0.0269) | 0.159 | (0.0439) |
| C_{tau, D14} | 0.589 | (0.6138) | 0.350 | (0.1026) | 0.402 | (0.4151) |
| C_{trough}, _{D7} | 0.0800 | (0.07521) | 0.0380 | (0.09308) | 0.1600 | (0.04163) |
| C_{trough, D14} | 0.5642 | (0.59619) | 0.2498 | (0.11455) | 0.4433 | (0.48780) |
| C_{avg, D7} | 0.237 | (0.0198) * | 0.222 | (0.0563) | 0.234 | (0.0741) |
| C_{avg, D14} | 1.004 | (1.0709) | 0.419 | (0.1792) | 0.634 | (0.6830) |
| Fluct, _{D7} | 241.104 | (274.2705) * | 137.091 | (119.2891) | 73.030 | (16.5213) |
| Fluct, _{D14} | 107.455 | (40.9046) | 72.165 | (30.0820) | 99.562 | (62.2334) |
| Accumulation Index, D7 | 3.288 | (0.8890) *** | 2.145** | | 2.091 | (0.9593) |
| Accumulation Index, D14 | 2.211 | (1.1208) | 2.204 | (0.4837) | 3.452 | (2.3159) |

* n = 3,
**n = 1,
*** n = 2

### 14.6.2—Skin Biopsy

Skin biopsy results were available for WinF90 (Cohort 2) and WinFB100 (Cohort 3) only. For both formulations, pirenzepine was detected at a higher level in the upper layer of the treated skin (calf) biopsy sample (predominated by the epidermis and dermis), when compared to the lower (dermis and adipose) layer. Like the plasma dataset, a large amount of inter-subject variability was noted in the skin biopsy concentration data in both cohorts.

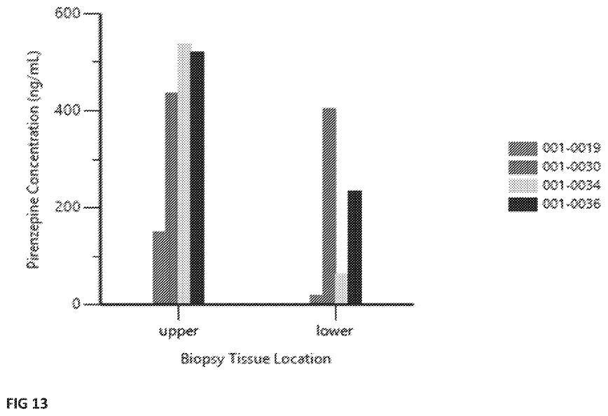

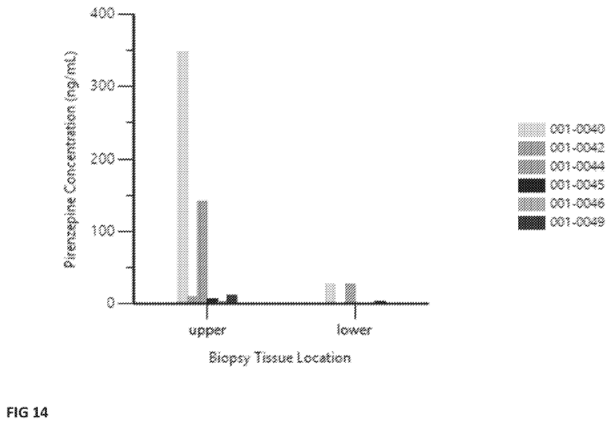

For WinF90/Cohort 2, there was a 6-fold greater concentration gradient from top to bottom of the treated skin, likely driven by flux of pirenzepine from a topical delivery system; and a 2-fold greater concentration gradient from top to bottom of the untreated (thigh) skin. The occurrence of pirenzepine in untreated skin could be a function of the physicochemical properties of the drug substance rather than the drug formulation. Pirenzepine is relatively hydrophilic (Log P=0.6, Solubility: 0.0682 mg/mL) (see pubchem.ncbi.nlm.nih.gov/compound/4848#section=MeSH-Entry-Terms accessed August 27) and as such would not be expected to accumulate in the fatty adipose layer of the lower layer of the skin biopsy, but rather preferentially accumulate in the interstitial fluid in the dermis, in accordance with some embodiments. See Groenendaal W, von Basum G, Schmidt K et al. Quantifying the composition of human skin for glucose sensor development. J. Diabetes and Technology, 2010, 4(5), 1032-1040. For this same reason, distribution into untreated skin from the bloodstream would also not be expected to be high in some embodiments (Table 13, FIGS. 13 and 14).

For WinFB100/Cohort 3, there was a 14 fold greater concentration gradient from top to bottom of the treated skin, and a 9-fold greater concentration gradient from top to bottom of the untreated skin. Interestingly, a subject's plasma exposure did not correlate with the relative pirenzepine concentration in the skin for either formulation. For example, WinF90, the formulation used in Cohort 2, may be more likely to retain pirenzepine in the skin, as indicated by a greater quantity of pirenzepine in the lower layer of the biopsy skin sample. However, this was not observed for WinFB100 dosed in Cohort 3, where less pirenzepine was retained in the lower level.

TABLE 13
Pirenzepine Biopsy Concentrations for Treated and Untreated Skin

| | | Plasma | | | | |
|---|---|---|---|---|---|---|
| | | Pirenzepine. | Treated | Treated | Untreated | Untreated |
| | | 8 h post dose | Calf Upper | Calf Lower | Thigh Upper | Thigh Lower |
| Subject | Formulation | Day 14 (ng/mL) | Half (ng/mL) | Half (ng/mL) | Half (ng/mL) | Half (ng/mL) |
| 001-0036 | WinF90 | 0.539 | 5310 | 1720 | 521 | 233 |
| 001-0019 | WinF90 | 0.263 | 14300 | 1810 | 150 | 20.8 |
| 001-0030 | WinF90 | 0.765 | 7340 | 1050 | 436 | 405 |
| 001-0034 | WinF90 | 0.329 | 12900 | 1740 | 536 | 64 |

| | | | | | | | | | | |
|---|---|---|---|---|---|---|---|---|---|---|
| Mean (SD) | 0.474 | (0.2269) | 9962.5 | (4319.19) | 1580.0 | (355.4) | 410.8 | (179.32) | 180.7 | (175.34) |

| Min, Max | | 0.263, 0.765 | 5310, 14300 | 1050, 1810 | 150, 536 | 20.8, 405 |
|---|---|---|---|---|---|---|
| Median | | 0.434 | 10120.0 | 1730.0 | 478.0 | 148.5 |
| 001-0042 | WinFB100 | 2.07 | 10300 | 1670 | 9.97 | 0 |
| 001-0040 | WinFB100 | 0.497 | 4620 | 245 | 349 | 27.0 |
| 001-0045 | WinFB100 | 0.162 | 5520 | 159 | 7.04 | 0 |
| 001-0046 | WinFB100 | 0.146 | 10200 | 256 | 4.16 | 0 |
| 001-0049 | WinFB100 | 0.715 | 3160 | 210 | 11.9 | 3.93 |
| 001-0044 | WinFB100 | 0.855 | 1610 | 53.4 | 142 | 28.3 |

| | | | | | | | | | | |
|---|---|---|---|---|---|---|---|---|---|---|
| Mean (SD) | 0.741 | (0.711) | 5901.67 | (3620.621) | 432.2 | (601.86) | 87.35 | (138.92) | 9.87 | (13.861) |

| Min, Max | 0.146, 2.07 | 1610, 10300 | 53.4, 1670 | 4.16, 349.0 | 0, 28.3 |
|---|---|---|---|---|---|
| Median | 0.606 | 5070 | 227.50 | 10.94 | 1.965 |

Approximate biopsy collection time 8 h post dose on Day 14

Plasma profiles of pirenzepine and concentrations in treated and untreated skin biopsy samples following 14 Days of topical application of three different pirenzepine formulations (WinFB34, WinF90 and WinFB100) have been determined according to protocol WST-PZP-001.

Following daily administration to the skin, pirenzepine was detectable in plasma from Day 1 in some, but not all, subjects (Cohort 1: 001-0002, 001-0005, 001-0013; Cohort 2: 001-0026; Cohort 3: 001-0040, 001-0044, 001-0049). By Day 7, AUC0-tau, D7 was similar for all cohorts, however the relative bioavailability criteria could not be satisfied due to low subject numbers and high variability. Following a doubling of the dose at Day 8, AUC0-tau, D14 was doubled. By Day 14, all subjects in all cohorts had quantifiable drug levels. However, a nonparametric analysis of Tmax, D1, Tmax, D7 and Tmax, D14 for each cohort did not find any significant differences between the formulations. Mean t½ across the three cohorts and formulations ranged from approximately 27 to 48 h and the accumulation index was approximately 2. Given the amount of concentration data variability and the low subject numbers, no PK generalizations could be made with regard to gender and demographics.

Skin biopsy results from Cohorts 2 and 3 indicated that pirenzepine preferentially was retained in the epidermis/dermis area of the skin (upper layer of the biopsy sample), with the lower layer of the biopsy sample (dermis and adipose tissue), retaining less of the dose. However, all layers of the skin, both treated and untreated, maintained a higher concentration of pirenzepine, when compared to the plasma concentrations.

### Example 15: Simplification of Pirenzepine Free Base Formulations to Enable Technical Transfer and Scale-Up

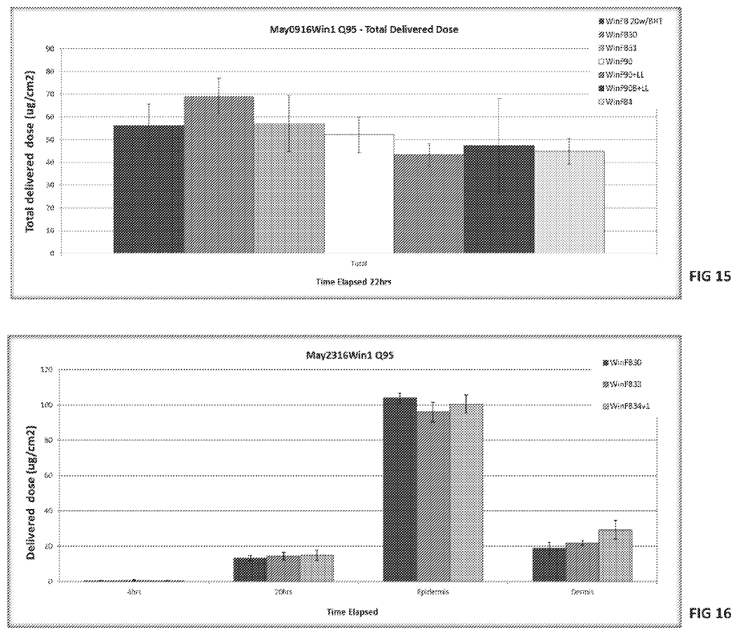

Formulation optimization were performed to simplify the lead formulations (WinF84 and WinFB20) to enable technical transfer and scale up. Due to the add-on layering approach to formulation development taken previously, it was hypothesized that the components of the lead formulations could be simplified to a certain degree, in support of scale-up activities. This simplification of vehicle components was shown not to have any impact on the flux of the formulations. The formulation ingredients are shown in Table 14 and Table 15. The results of this flux study are shown in FIG. 15 and FIG. 16.

Skin biopsies were collected at 22 hours post topical administration and the total delivered dose was determined (FIG. 15).

Skin biopsy results showed that embodiments of the formulations disclosed herein were preferentially retained in the epidermis/dermis area of the skin (upper layer of the biopsy sample), whereas the lower layer of the biopsy sample (dermis and adipose tissue) retained less of the dose (FIG. 16). Also shown are the total delivered dose at 4 hours and 20 hours post administration of the formulations.

TABLE 14
Simplification of pirenzepine dihydrochloride salt and pirenzepine free base formulations

| | Formulation name | | | | | | |
|---|---|---|---|---|---|---|---|
| | WinFB 20 w/BHT | WinFB30 | WinFB31 | WinF90 | WinF90 + LL | WinF90B + LL | WinF84 |
| Formulation Notes | | | | | | | |
| Dosing (ul): | 5.0 | 5.0 | 5.0 | 5.0 | 5.0 | 5.0 | 5.0 |
| wt % Pirenzepine | 4 | 4 | 4 | 7.75 | 7.75 | 7.75 | 7.72 |
| Ingredient | wt/wt % | wt/wt % | wt/wt % | wt/wt % | wt/wt % | wt/wt % | wt/wt % |
| Pirenzepine free base | 4 | 4 | 4 | | | | |
| Water | 8 | 8 | | | | | |
| DMSO | 16 | 16 | 16 | | | | |
| Brij S20 | 3.2 | | | | | | |
| Brij L4 | 3.2 | 3.2 | 3.2 | | | | |
| Lauryl lactate | 1.6 | 1.6 | 1.6 | | | | |
| Ethanol | 32 | 33.6 | 37.6 | | | | |
| Propylene glycol | 31.9 | 33.5 | 37.5 | | | | |
| BHT | 0.1 | 0.1 | 0.1 | | | | |
| Pirenzepine Dihydrochloride | | | | 7.75 | 7.75 | 7.75 | 7.72 |
| Water | | | | 44 | 44 | 44 | 42.67 |
| NaOH | | | | pH 5.5 | pH 5.5 | pH 5.5 | 0.74 |
| Citric Acid | | | | 0.41 | 0.41 | 0.41 | 0.23 |
| Sodium citrate, dihydrate | | | | 2.22 | 2.22 | 2.22 | 1.25 |
| DMSO | | | | 12 | 12 | 12 | 10.47 |
| Brij S20 | | | | 2.75 | 2.75 | | 2.79 |
| Brij L4 | | | | 2.75 | 2.75 | 2.75 | 2.79 |
| Ethanol | | | | 20 | 20 | 20 | 18.98 |
| Propylene glycol | | | | 7 | 7 | 7 | 6.98 |
| Glycerine | | | | | | | 2.46 |
| Lauryl lactate | | | | | 1.5 | 1.5 | |
| Benzyl Alcohol | | | | | | | 2.09 |
| BHT | | | | 0.05 | 0.05 | 0.05 | 0.05 |

TABLE 15
Simplification of pirenzepine free base formulations

| | Formulation name | | |
|---|---|---|---|
| | WinFB30 | WinFB33 | WinFB34v1 |
| Ingredient | wt/wt % | wt/wt % | wt/wt % |
| Pirenzepine base | 4 | 4 | 4 |
| Water | 8 | 8 | 8 |
| Dimethyl sulfoxide | 16 | 16 | 16 |
| Brij L4 | 3.2 | 3.2 | 3.2 |
| Lauryl lactate | 1.6 | | |
| Isopropyl myristate | | 2 | |
| Crodamol GTCC | | | 2 |
| Ethanol | 33.6 | 33.4 | 33.4 |
| Propylene glycol | 33.5 | 33.3 | 33.3 |
| BHT | 0.1 | 0.1 | 0.1 |

### Example 16: Screening Pirenzepine Free Base Formulations to Improve Drying Time and to Increase Viscosity

Formulation optimization was performed to improve the drying rate of the formulation and increase the formulation viscosity to improve ease of application in support of longer-term clinical trial use. Winfb34 placebo formulations were prepared covering a range of changes to the winfb34 vehicle, with particular focus on lowering the propylene glycol concentration, which was believed to be responsible for the extending drying time. Based on feedback (including cosmetic feel and drying time) from a panel of test subjects, the winfb34 vehicle was altered accordingly. A series of four formulations containing pirenzepine base were then tested against the winfb34 formulation, along with a gelled version of winfb34. The formulation ingredients are shown in table 16. The results of this flux study are shown in FIG. 17.

Skin biopsy results showed that embodiments of the formulations disclosed herein were preferentially retained in the epidermis/dermis area of the skin (upper layer of the biopsy sample), whereas the lower layer of the biopsy sample (dermis and adipose tissue) retained less of the dose (FIG. 17). Also shown are the total delivered dose at 3 hours, 6 hours, and 24 hours post administration of the formulations.

TABLE 16
Pirenzepine free base formulations modified to improve drying time

| | Formulation name | | | | | |
|---|---|---|---|---|---|---|
| | WinFB34 | WinFB34gel | WinFB50 | WinFB51 | WinFB52 | WinFB53 |
| Ingredient | wt/wt % | wt/wt % | wt/wt % | wt/wt % | wt/wt % | wt/wt % |
| Pirenzepine Base | 4.0 | 4.0 | 4.0 | 4.0 | 4.0 | 4.0 |
| Water | 4.0 | 4.0 | | | | |
| Dimethyl Sulfoxide, USP | 16.0 | 16.0 | 16.0 | 16.0 | 16.0 | 16.0 |
| Capric/Caprylic Triglyceride, NF, | 2.0 | 2.0 | 10.0 | 10.0 | 5.0 | 5.0 |
| Laureth-4, (PhEur), Brij L4 | 3.0 | 3.0 | 3.0 | 3.0 | 3.0 | 3.0 |
| Ethanol, USP | 35.5 | 34.3 | 57.0 | 55.5 | 56.0 | 54.5 |
| Propylene Glycol, USP | 35.5 | 34.3 | 10.0 | 10.0 | 15.0 | 15.0 |
| Cyclomethicone | | 1.0 | | | 1.0 | 1.0 |
| HY119 | | 1.5 | | 1.5 | | 1.5 |

### Example 17: Screening Pirenzepine Free Base Formulations to Improve Drying Time and to Increase Viscosity without the Addition of Cyclomethicone

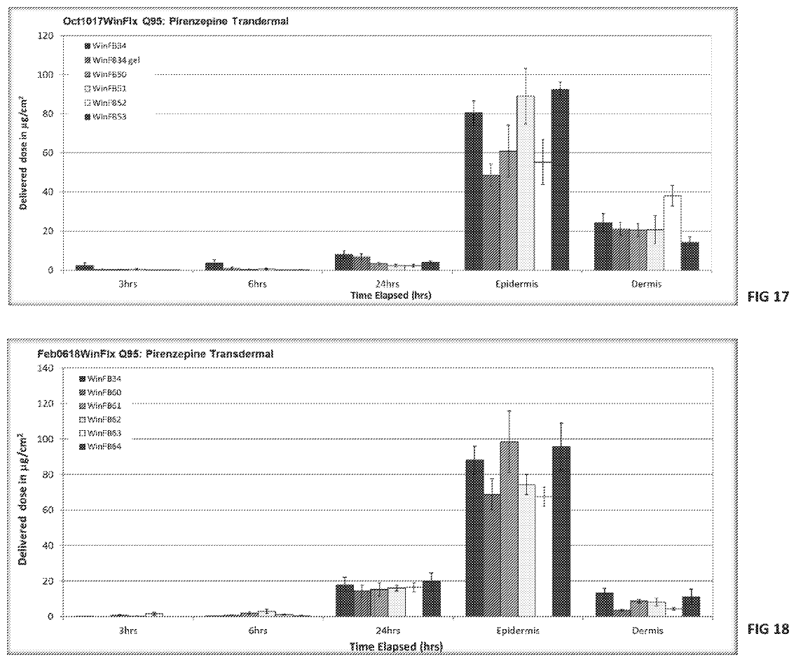

Formulation optimization was performed to improve the end-user appeal (i.e. improve drying time, increase viscosity slightly) and remove the cyclomethicone specific to WinFB53. A series of five formulations containing pirenzepine base were then tested against the WinFB34 lead formulation (without BHT for simplicity). The formulation ingredients are shown in Table 17. The results of this flux study are shown in FIG. 18.

Skin biopsy results showed that embodiments of the formulations disclosed herein were preferentially retained in the epidermis/dermis area of the skin (upper layer of the biopsy sample), whereas the lower layer of the biopsy sample (dermis and adipose tissue) retained less of the dose (FIG. 18). Also shown are the total delivered dose at 3 hours, 6 hours, and 24 hours post administration of the formulations.

TABLE 17
Pirenzepine Free Base Formulations modified to decrease drying
time and increase viscosity, without cyclomethicone

| | Formulation name | | | | | |
|---|---|---|---|---|---|---|
| | WinFB34 | WinFB60 | WinFB61 | WinFB62 | WinFB63 | WinFB64 |
| Ingredient | wt/wt % | wt/wt % | wt/wt % | wt/wt % | wt/wt % | wt/wt % |
| Pirenzepine Base | 4.0 | 4.0 | 4.0 | 4.0 | 4.0 | 4.0 |
| Water | 4.0 | | | | | |
| Dimethyl Sulfoxide, USP | 16.0 | 12.0 | 16.0 | 16.0 | 16.0 | 16.0 |
| Capric/Caprylic Triglyceride | 2.0 | 5.0 | 20.0 | 15.0 | 10.5 | 10.0 |
| Laureth-4, (PhEur), Brij L4 | 3.0 | 3.0 | 3.0 | 3.0 | 3.0 | 3.0 |
| Ethanol, USP | 34.8 | 42.5 | 40.5 | 45.5 | 47.0 | 42.5 |
| Propylene Glycol, USP | 34.8 | 32.0 | 15.0 | 15.0 | 18.0 | 15.0 |
| Cetyl alcohol | | | | | | 8.0 |
| HY119 | 1.5 | 1.5 | 1.5 | 1.5 | 1.5 | 1.5 |

### Example 18: Human Results

### Example 18. Treatment of Subject with Diabetic Peripheral Neuropathy

Men and women having peripheral neuropathy are screened based on peripheral neuropathy induced by diabetes (type 1 or type 2), chemotherapy, HIV, surgery, or other insults or injuries. Patients can be screened to assess the appearance of the skin and elasticity, peripheral appendage strength and flexibility, gait, ankle reflexes, and any presence of ulceration, and Semmes Weinstein filament testing is performed. In some cases, electromyography, ultrasound therapy and/or blood testing can also be performed prior to initiation of the study. Symptomology will depend upon the type of peripheral neuropathy being treated, but will typically include pain, numbness, tingling, imbalance, fasciculations, muscle cramping or loss of sensation. Many of the subjects may have previously been treated with Cymbalta®, Lyrica® or Nucynta®. A skin punch biopsy is obtained from the calf and/or ankle of a leg of each subject using standard procedures as follows.

This study was to evaluate the efficacy of WST-057 to increase IENFD present in punch biopsies collected in the calf. The Intraepidermal Nerve Fiber Density will be counted in these punch biopsies and the change in the density will be measured between screening and the end of each visit. The more nerves or a higher nerve density correlates with better sensation.

Norfolk Quality of Life Measure (QOL) Patient Questionnaire

This will be performed to determine the impact of once-daily topical dosing of WST-057 on a QOL measurement. The score from the QOL questionnaire will be tabulated from each visit to determine if an improvement in the quality of life could be measured using this scale between screening and the week 24 visit. All symptoms (1-7) are scored as either a 1 or a 0, indicating a presence or absence of the symptom. With the exception of Questions 31, and 32, the other items are scored according to the 5-point Likert Scale (0-4, “No Problem” to “Severe Problem”). In Question 31, “Good”, the middle item, is scored as 0. “Very Good” is scored as −1, Excellent” is scored as −2. “Fair is scored as 1, and “Poor” is scored as 2. In Question 32, “About the Same”, the middle item, is scored as 0. “Somewhat better” is scored as −1, “Much better” is scored as −2. “Somewhat worse” is scored as 1, and “Much worse” is scored as 2. A higher score indicates worse neuropathy-related quality of life score than a lower number.

Other Outcome Measures:

Quantitative Thermal Threshold (QST) Quantitative Vibration Threshold (QVT)

Determine the impact of once daily dosing of WST-057 on QST and QVT. Sensory and vibration probes attached to an instrument that measures response will be applied to the feet of patients to determine if there is an improvement from the screening visit until 24 weeks post dosing. The lower the threshold for thermal and vibration sensation indicates better feeling or less neuropathy.

Subjects were instructed to apply the invention topical solution to their feet once each day at the same time, usually prior to going to sleep, and depending on their severity into their calf.

After one or two months of treatment, symptomatic relief is achieved in most subjects. Some subjects with more severe symptomology require more weeks of treatment before achieving symptomatic relief. A 3 mm skin punch biopsy is obtained at baseline and within three to six months of treatment depending upon the subject's response. The control biopsy and the post-treatment biopsy are analyzed to determine epidermal nerve fiber density, and compared to determine the effectiveness of treatment.

Examples 18.1 through 18.6 are illustrative of successful use of a topical composition comprising 4% pirenzepine in the invention solution in treating peripheral neuropathy. The topical composition used in each of the Examples used DMSO as the low volatility solvent and polyethylene glycol alkyl ether as the a polyether surfactant, and the concentration of pirenzepine in the topical compositions used was 4% by weight based on the total weight of the topical composition.

### Example 18.1

Diabetic Peripheral Neuropathy—Type 1

A 59 year old Caucasian male with type 1 diabetes from age 20 began suffering from involuntary muscle movement in calves for 2 years. Over the next six months, subject had several health issues including sharp pains in feet, muscle cramping in calves, aching feet and legs, balance issues and lost the ability to perform any leg exercises. He was diagnosed with diabetic peripheral neuropathy via an EMG test. Patient also suffered from fasciculations in calves which impeded normal sleep. Patient began applying 4% pirenzepine solution on his feet and legs prior to going to sleep. Within 3 weeks, subject noticed fasciculations were not as severe and was able to sleep. Eventually, other symptoms were mitigated over the next several months of use.

### Example 18.2

Diabetes Mellitus Insulin Dependent Peripheral Neuropathy

A 52-year old black male subject was suffering for several years from pain and numbness of both feet due to Peripheral Neuropathy secondary to insulin dependent Diabetes mellitus. Pain was so severe that sleep was denied from chronic pain. Even conventional pain medicines did not help. Pirenzepine 4% solutions was applied daily by the patient to his skin near the painful area. Within 3 months, subject experienced pain and numbness relief in both feet. Thereafter the subject achieved restful sleep. Subject continues to use the solution for relief successfully.

### Example 18.3

Surgically Induced Peripheral Neuropathy (HNP)

A 72-year-old Caucasian female had carotid arterial surgery leaving a 6-inch scar on her neck and paralyzing nerves on left check for 18 months—loss of sensation. A 4% Pirenzepine solution was applied to her face daily for 3 weeks. Substantial senasation (˜90%) feeling (temperature and light touch) has returned to facial area, and has remained despite cessation of the topical drug (6 months).

### Example 18.4

Prevention of Chemotherapy Induced Peripheral Neuropathy

A 54 year old Asian female with stage 3 endometrial carcinoma was treated with radio chemotherapy (cisplatin, then four rounds of carboplatin-taxol combination), She applied the pirenzepine topical solution a few days prior to chemotherapy treatments and continued applying. Patient felt the neuropathy affects after the first round of carboplatin. She developed pain and numbness of her feet and hands caused by peripheral neuropathy from chemotherapy for the first several days. Her peripheral neuropathy subsided. She continued to apply the pirenzepine topical solution to the skin of her feet and hands. The pain and numbness did not return in her hands and feet.

### Example 18.5

Chemotherapy Induced Peripheral Neuropathy

A 74-year-old Caucasian male was diagnosed with Large Basal Cell Non-Hodgkin Lymphoma. He was treated with R-CHOP therapy that included Vincristin and high doses of Prednisone, six treatments were administered three weeks apart. About halfway through the chemotherapy cycles, he experienced extreme weakness in his legs to the point where I could not rise from a seated position. Following the chemotherapy course, his feet became numb, and his balance was affected. Exercising the foot regained control of his feet but not the feeling. The skin of the feet, up to the sock line, and my fingertips were completely numb. Subject began applying 4% pirenzepine solution after showering. After several months, feeling and tingling returned, including minor pain. Balance also improved. After several additional months, pain subsided and improved sensation and balance.

### Example 18.6

Idiopathic Peripheral Neuropathy

A 76 year old Caucasian female began feeling numbness and tingling in her feet and hands due to unknown idiopathic reasons, She was unable to have the sense to avoid objects with her feet, with increased imbalance. She began applying a 4% pirenzepine solution daily. After a month of use, she began to increase sensation. She also went to physical therapy after the first month, where electrical stimulation was applied. Therapist noted that she was able to feel the stimulation at a level 8, whereas before application of the solution, she could not feel the stimulation until it was turned up to a level 25. Subject also noticed for first time the gel pack applied to her during treatment was heated. After several months, she no longer had the imbalance or ran into objects with her feet. Although she noticed no significant additional changes, she noted that a week of non-use would significantly deteriorate condition.

1. A topical formulation comprising: (i) a muscarinic acetylcholine receptor antagonist or a salt or derivative, (ii) a low volatility solvent, and (iii) a polyalkylene glycol alkyl ether.

2. The topical formulation of claim 1, wherein the muscarinic acetylcholine receptor antagonist is selected from the group consisting of: pirenzepine, pirenzepine free base, and a pirenzepine salt.

3. The topical formulation of claim 1, wherein said low volatility solvent is DMSO.

4. The topical formulation of claim 1, wherein said polyether surfactant is a polyethylene glycol alkyl ether.

5. The topical formulation of claim 1, further comprising a fatty acid ester.

6. The topical formulation of claim 5, wherein the fatty acid ester is lauryl lactate.

7. The topical formulation of claim 1, further comprising a capric triglyceride.

8. The topical formulation of claim 1, further comprising benzyl alcohol.

9. The topical formulation of claim 1, further comprising dimethyl isosorbide.

10. The topical formulation of claim 1, wherein the muscarinic acetylcholine receptor antagonist is pirenzepine salt, and the composition comprises less than 20% pirenzepine salt.

11. The topical formulation of claim 2, wherein the muscarinic acetylcholine receptor antagonist is pirenzepine free base, and the composition comprises less than 10% pirenzepine free base.

12. The topical formulation of claim 11, wherein the topical formulation comprises about between about 1-5% pirenzepine free base.

13. The topical formulation of claim 1, wherein the topical formulation is selected from a gel, lotion, cream, ointment, and liquid formulation.

14. The topical formulation of claim 1, further comprising a muscle relaxant or cramping relief compound.

15. The topical formulation of claim 12, wherein the muscle relaxant or cramping relief compound is selected from a magnesium ion, vitamin B1, B2, B6 and/or vitamin B12.

16. The topical formulation of claim 1, further comprising a compound which aids in a person's sleep.

17. The topical formulation of claim 1, further comprising an anaesthetic or analgesic compound.

18. The topical formulation of claim 17, wherein said analgesic compound is an amino ester or amide.

19. The topical formulation of claim 17, wherein said analgesic compound is a cannabinoid.

20. The topical formulation of claim 1, wherein a drying time of the formulation ranges from about 30 seconds to about 30 minutes.

21. The topical formulation of claim 1, wherein a viscosity of the formulation ranges from about 300 mPa·s to about 15,000 mPa·s.

22. The topical formulation of claim 1, wherein the formulation comprises a dose of pirenzepine of about 40 μg/cm² to about 120 μg/cm².

23. The topical formulation of claim 22, wherein the formulation is a dose of pirenzepine of about 40 μg/cm² to about 120 μg/cm² formulated to be delivered via the epidermis.

24. The topical formulation of claim 23, wherein the formulation is configured to be delivered to the epidermis within about 18 hours to about 24 hours.

25. The topical formulation of claim 24, wherein the formulation is configured to be delivered via the epidermis within about 22 hours.

26. A method of inhibiting, ameliorating, reducing a severity of, treating, delaying the onset of, or preventing one or more symptoms peripheral neuropathy in a subject, comprising topically administering the topical formulation of claim 1 to the subject.

27. The method of claim 26, wherein the peripheral neuropathy is selected from the group consisting of type I diabetic peripheral neuropathy, type II diabetic peripheral neuropathy, diabetes mellitus insulin dependent peripheral neuropathy, surgically induced peripheral neuropathy, chemotherapy induced peripheral neuropathy, infectious disease induced peripheral neuropathy, and idiopathic peripheral neuropathy.

28. The method of claim 27, wherein the infectious diseases is HIV.
